(19) United States
(12) Patent Application Publication
(10) Pub. No.: US 2023/0355242 A1
(43) Pub. Date: Nov. 9, 2023

(54) ULTRASOUND CONTROLLED REMOTELY DEFLATABLE ENDOSCOPIC DETACHABLE BALLOON

(71) Applicants: California Institute of Technology, Pasadena, CA (US); The Regents of the University of California, Oakland, CA (US)

(72) Inventors: Max KUDISCH, Pasadena, CA (US); Jeong Hoon KO, Pasadena, CA (US); Chiara DARAIO, Pasadena, CA (US); Mikhail G. SHAPIRO, Pasadena, CA (US); Di WU, Pasadena, CA (US); Gunho KIM, Pasadena, CA (US); Robert H. GRUBBS, South Pasadena, CA (US); Michael R. HARRISON, San Francisco, CA (US); Sunghoon KIM, Oakland, CA (US)

(21) Appl. No.: 18/096,256

(22) Filed: Jan. 12, 2023

Related U.S. Application Data
(60) Provisional application No. 63/299,514, filed on Jan. 14, 2022.

(51) Int. Cl. A61B 17/12

(52) U.S. Cl. CPC A61B 17/12136; A61B 17/1204; A61B 17/12031

(57) ABSTRACT

Aspects disclosed herein include an implantable, detachable, and removable medical device comprising: a therapeutic balloon configured to perform a therapeutic activity inside a living subject; and a focused-ultrasound activatable actuator operably connected to the therapeutic balloon; wherein the actuator is capable of being activated between a closed state and an open state remotely via a focused ultrasound beam; wherein the device is configured to be in an implantation state to facilitate implantation of the device in the living subject, in a therapeutic state to facilitate performance of the therapeutic activity, and subsequently in an expulsion state to facilitate expulsion of the medical device from the living subject; and wherein the implantation state, therapeutic state, and expulsion state are different from each other

### CROSS-REFERENCE TO RELATED APPLICATIONS

This application claims the benefit of priority to U.S. Provisional Patent Application No. 63/299,514, filed Jan. 14, 2022, which is hereby incorporated by reference in its entirety.

### BACKGROUND OF INVENTION

Congenital diaphragmatic hernia is a neonatal disease characterized by incomplete development of diaphragm, causing abdominal content such as the intestine, spleen, stomach, and liver to enter the chest cavity. Space occupation of the chest with abdominal viscera prevents proper development of both ipsilateral and contralateral lung. Neonates born with severe hypoplastic lung will die from inadequate oxygenation without medical and surgical interventions. Congenital diaphragmatic hernia can be due to genetic disorder but often occurs without any identifiable genetic cause.

The current standard treatment for congenital diaphragmatic hernia is to provide adequate oxygenation and ventilation to the newborn lung. Airway control with endotracheal tube intubation is needed if the newborn's respiratory need is severe. If conventional oxygenation support is inadequate, extracorporeal membrane oxygenation (ECMO) may be used. Surgical intervention to reduce the abdominal content from the chest into the abdominal domain and to repair the diaphragm hernia is undertaken once the neonate is hemodynamically stable. Ultimate survival of the neonate is dependent on the degree of hypoplasia or lack of lung development during fetal development. During prenatal surveillance, if the fetus is found to have extremely poor lung development and the chances of survival upon birth is predicted to be very low, fetal intervention can be offered. Fetoscopic endoluminal tracheal occlusion (FETO) is a surgical method utilized to promote fetal lung growth. During fetal development, pulmonary fluid from the lung passes through the trachea and is expelled into the amniotic space. By occluding the trachea, the pulmonary fluid is forced to accumulate within the trachea causing back pressure into the lung tissue. This increased pressure stimulates lung growth during the period of tracheal occlusion. Two FETO procedures are done; first, tracheal occlusion is established, and second, the trachea is reopened at a later time. The first procedure is done at 27 weeks gestation. An incision is made on the mother's abdomen. Through the incision, an access port is placed into the uterus. An endoscope is guided through the port, into the mouth of the fetus, then into the fetal trachea. Through the channel within the endoscope, an endoscopic detachable balloon catheter is passed into the trachea. The detachable balloon is positioned within the mid-trachea under endoscopic vision. After fluid inflation of the balloon, the delivery catheter is detached from the balloon. The scope and the catheter are both removed from the uterus. A second procedure to deflate the balloon is done at 34 weeks gestation. The deflation of the balloon is accomplished by passing a needle through the mother's abdomen and into the uterus under ultrasound guidance. If the tracheal balloon can be visualized with ultrasound probe and the trajectory of the needle path is safe, the balloon can be punctured with the needle. Upon balloon deflation, it is expelled out from the trachea by accumulated lung pressure. If the fetal position does not allow needle deflation of the balloon, endoscopic maneuver is done like the first procedure. The balloon is endoscopically punctured and removed from the trachea. Once the baby is delivered at full term, standard medical and surgical treatments are rendered.

FETO procedure complications can be divided into intra-operative and post-procedure complications. Examples of intraoperative complications include bleeding from accidental injury to the placenta causing fetal demise and difficulty with tracheal balloon deflation. Post-procedure complications include initiation of preterm labor and inadvertent premature delivery.

Given the fatal complications that may occur with the second FETO procedure, there is a need in the art for remote deflation of the tracheal balloon without surgical intervention.

### SUMMARY OF THE INVENTION

Provided herein are medical devices that address at least the issues described above, such as remote deflation of a tracheal balloon without invasive surgical procedures being necessarily. Also provided herein are methods of making the medical devices, methods of using the medical devices, and methods of treating a condition in a living subject using the medical devices disclosed herein. The medical devices disclosed herein include a therapeutic balloon, such as a tracheal balloon useful for a FETO procedure, and an actuator that can be remotely activated or actuated using focused ultrasound. In various aspects, the device has a therapeutic state characterized by the therapeutic balloon being inflated, such as to provide the therapeutic activity/function of tracheal occlusion. In various aspects, the actuator is in a closed state during the therapeutic activity, such as tracheal occlusion, in which case a fluid is retained in the balloon thereby keeping the balloon inflated to maintain occlusion. In various aspects, the medical device may remain implanted in a living subject in the therapeutic state, neither directly nor indirectly physical connected to any component, device, or accessory that is external of the living subject, for a prolonged period of time, such as at least several weeks. In various aspects, the actuator can be remotely activated via a focused ultrasound beam, which is non-invasively directed at/into the living subject, which, in various aspects, activates or actuates the open state of the actuator, thereby allowing fluid to drain out of the therapeutic balloon and further thereby deflating the balloon, which corresponds to the expulsion state of the device. In various aspects, once the therapeutic balloon is deflated, and the medical device is therefore in its expulsion state, the therapeutic balloon no longer provides occlusion and may be expelled naturally by the body or natural bodily process(es) of the living subject. As such, the medical devices disclosed herein can be used for FETO procedures but without needing the second procedure of having the device or balloon invasively removed, thereby making FETO procedures safer and potentially expanding the eligibility for the procedure to save more lives.

Aspects disclosed herein include an implantable, detachable, and removable medical device comprising: a therapeutic balloon configured to perform a therapeutic activity inside a living subject; and a focused-ultrasound activatable actuator operably connected to the therapeutic balloon; wherein the actuator is capable of being activated between a closed state and an open state remotely via a focused ultrasound beam; wherein the device is configured to be in an implantation state to facilitate implantation of the device in the living subject, in a therapeutic state to facilitate performance of the therapeutic activity, and subsequently in an expulsion state to facilitate expulsion of the medical device from the living subject; and wherein the implantation state, therapeutic state, and expulsion state are different from each other.

Aspects disclosed herein include a method for treating a condition in a living subject, the method comprising: implanting a medical device with the aid of an implant-accessory; filling a therapeutic balloon with a biologically-benign fluid via the implant-accessory; and detaching the implant-accessory from the medical device; wherein the medical device is implantable, detachable, and removable and the medical device comprises: a therapeutic balloon configured to perform a therapeutic activity inside a living subject; and a focused-ultrasound activatable actuator operably connected to the therapeutic balloon; wherein the actuator is capable of being activated between a closed state and an open state remotely via a focused ultrasound beam; wherein the device is configured to be in an implantation state to facilitate implantation of the device in the living subject, in a therapeutic state to facilitate performance of the therapeutic activity, and subsequently in an expulsion state to facilitate expulsion of the medical device from the living subject; and wherein the implantation state, therapeutic state, and expulsion state are different from each other.

Aspects disclosed herein include a method for making the medical device of any one of the preceding claims, the method comprising: operably attaching a polymer component having an ultrasound-absorbing shape memory polymer to the therapeutic balloon thereby forming the focused-ultrasound activatable actuator; wherein the medical device is implantable, detachable, and removable and the medical device comprises: a therapeutic balloon configured to perform a therapeutic activity inside a living subject; and a focused-ultrasound activatable actuator operably connected to the therapeutic balloon; wherein the actuator is capable of being activated between a closed state and an open state remotely via a focused ultrasound beam; wherein the device is configured to be in an implantation state to facilitate implantation of the device in the living subject, in a therapeutic state to facilitate performance of the therapeutic activity, and subsequently in an expulsion state to facilitate expulsion of the medical device from the living subject; and wherein the implantation state, therapeutic state, and expulsion state are different from each other.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIGS. 1A-1D: FIG. 1A is a depiction of a medical device, according to various aspects herein, having a therapeutic balloon (“balloon”) and an actuator (“deflation valve”). The drawing shows the medical device in process of obtaining the therapeutic state, according to various aspects, as or after the balloon is inflated and before the implant-accessory (“catheter” and “catheter needle”) is disconnected at the locking mechanism (“inflation valve”), according to various aspects herein. The drawing shows the actuator having a polymer plug, according to aspects of a polymer component, comprising a shape memory polymer. In various aspects, the medical device comprises an actuator-securing mechanism (“security wire”) to maintain connection between the therapeutic balloon and the actuator in the implantation, therapeutic, and expulsion states of the device. FIG. 1B is a schematic of a medical device in its implantation state, according to aspects herein. FIG. 1C is a schematic of a medical device in its therapeutic state, according to aspects herein. FIG. 1D is a schematic of a medical device in its expulsion state, according to aspects herein.

FIGS. 2A-2D: Schematics of various non-limiting aspects of an actuator and a polymer component or a plug thereof, also referred to herein as Valve Concepts 1-4. Specific steps, descriptors, temperatures, and proportions, for example, are shown merely as illustrative examples and are not intended to be limiting of the aspects disclosed herein. FIG. 2A: Schematic of a simplified proof-of-concept balloon valve. One concept for a shape changing valve plug is illustrated in which the plug expands and is ejected from the valve. FIG. 2B: A tall cylinder is compressed to form a temporary shape which has a larger diameter. When used to plug the valve end, contraction of the diameter upon heat or ultrasound trigger opens the valve. FIG. 2C: Compression closes a hole to form a flat membrane to seal the valve end. Shape memory leads to reopening of the hole. FIG. 2D: The concept of a valve plug which unfolds to reveal a hole is illustrated. The plug is attached to the valve end with the hole facing the valve.

FIG. 3: Photographs showing heat induced opening of a proof-of-concept actuator, according to various aspects herein. Photographs of shape memory plug as it is heated with a heat gun to Tₜᵣₐₙₛ, at which point it turns translucent and expands. As it expands it is forced out of the valve end and is ejected after 17.1 seconds, for example.

FIG. 4: A sequence of photographs showing a shape change of a polymer component, having a shape memory polymer, from a temporary shape (at t=0 s) to a permanent shape (at t=90.0 s) thereof. A compressed cylindrical sample expands in height and contracts in diameter to restore its permanent shape. Time zero is when the sample is first placed on a dish heated to 60° C. The shape change demonstrated here is useful, for example, for Valve Concept 2 of FIG. 2B.

FIG. 5: A sequence of photographs showing an actuator, according to aspects herein, wherein a shape change of a polymer component, having a shape memory polymer, from a temporary shape corresponding to a closed state of the actuator to a permanent shape thereof corresponding to an open state of the actuator, according to various aspects herein. The shape change demonstrated here is useful, for example, for Valve Concept 2 of FIG. 2B. The valve, or actuator, opens upon exposure to heat. Photographs taken at various time points show the deflation process. Initially, the sample has a disc temporary shape which tightly fits the septum. After deflation, gaps denoted by white arrows appeared between the sample and septum. The shape change demonstrated here is useful, for example, for Valve Concept 2 of FIG. 2B.

FIG. 6: Photographs and associated exemplary details of a custom laser-cut clamp, according to various aspects herein, used to compress a shape memory polymer or a polymer component therewith.

FIG. 7: A sequence of photographs showing a shape change of a polymer component, having a shape memory polymer, from a temporary shape (at t=0.0 s) to a permanent shape thereof, according to various aspects herein. The temporary shape is hole-free and is capable of forming a fluidic or plug, whereas the permanent shape comprise a hole through which a fluid my flow. In various aspects herein, the temporary shape corresponds to a closed state of an actuator having the polymer component, such as depicted here, and the permanent shape corresponds to an open state of an actuator having the polymer component, such as depicted herein. The flattened sample is observed to thicken, opening a hole that was present in the permanent shape. The shape change demonstrated here is useful, for example, for Valve Concept 3 of FIG. 2C.

FIG. 8: A sequence of photographs showing a shape change of a polymer component, having a shape memory polymer, from a temporary shape (at t=0 s) to a permanent shape thereof (at t=30.0 s), according to various aspects herein. The temporary shape resembles a folded sheet, wherein a hole is fluidically sealed as a result of a portion of the sheet being folded over the hole. The shape change is an unfolding of the sheet, as a result of which the permanent shape, in contrast, comprises an unsealed hole through which a fluid my flow. In various aspects herein, the temporary shape corresponds to a closed state of an actuator having the polymer component, such as depicted here, and the permanent shape corresponds to an open state of an actuator having the polymer component, such as depicted herein. A folded sample with a masked hole undergoes shape memory unfolding when heated. The shape change demonstrated here is useful, for example, for Valve Concept 4 of FIG. 2D.

FIG. 9A: Schematic description of a setup for testing of ultrasound activation of an actuator or of an ultrasound-induced shape change. FIG. 9B: An exemplary plot showing a thermal camera temperature plotted vs. time for an ellipsoid region positioned on a polymer component sample. In various experiments, the temperature of the surrounding water did not change significantly from 37.0° C. FIG. 9C: Photographs of ultrasound testing of a folded shape memory polymer, such as one of FIG. 8 or one corresponds to FIG. 2D. Rapid unfolding is observed upon ultrasound exposure from the folded temporary shape (at t=0.0 s) to an unfolded permanent shape (at t=6.6 s). (“US”=ultrasound).

FIG. 10: Scheme and chart describing synthesis of a shape memory polymer, or polymer component therewith, according to various aspects disclosed herein. The polymer component optionally comprises additive particles in the shape memory polymer(s), according to various aspects herein. Fe₃O₄ nanoparticles are named by particle diameter. Additive density is obtained from product datasheets. Melting temperature, Tₘ, and crystallization temperature, T_{c}, are obtained from differential scanning calorimetry using the second cycle heating or cooling curve, respectively.

FIG. 11: An ¹H NMR spectrum of a crosslinking moiety, according to some aspects herein, of a shape memory polymer.

FIG. 12: An example differential scanning calorimetry (DSC) data used to determine T_{c} and Tₘ of a shape memory polymer. Stability of the DSC results over 3 cycles beyond the 1^{st} cycle is noted.

FIG. 13A: A plot of polymer sample thickness vs. water or equilibration temperature for a sample with a compressed temporary shape. FIG. 13B: DSC of a polymer sample with the same thermal history and processing as in FIG. 13A.

FIG. 14A: An exemplary scheme and table showing polymerization conditions and Tₘ and T_{c} values measured by DSC. FIG. 14B: Plot of Tₘ vs. crosslinking moiety loading.

FIG. 15A: Photograph of a completed 6×scale valve. The SMP plug can be seen inside the septum. A white dotted line marks a cutting plane for the cross section diagram (inset). Black dotted lines indicate a through hole blocked by the valve plug (gray). FIG. 15B: Photograph of the entire setup showing balloon with valve placed just at the water surface (end submerged) above the ultrasound transducer. The tubing in the photo connects to the circulating pump that keeps the transducer clear of bubble buildup. FIG. 15C: A close up photograph of the valve end right as ultrasound is turned on. FIG. 15D: A photograph after 10 s of ultrasound exposure. A bubble exiting the opened valve is denoted with the black arrow.

FIG. 16: Photograph of the polymer sample placed in coupling gel in the ultrasound setup.

### STATEMENTS REGARDING CHEMICAL COMPOUNDS AND NOMENCLATURE

In general, the terms and phrases used herein have their art-recognized meaning, which can be found by reference to standard texts, journal references and contexts known to those skilled in the art. The following definitions are provided to clarify their specific use in the context of the invention.

The term “implant accessory” refers to an accessory, component, feature, or tool that facilitates implantation of the medical device in a desired region or location of the living subject. For example, an implant accessory may allow for a user, such as a surgeon, to manipulate the positioning or otherwise move the medical device within the living subject from one location to another in order to ultimately position the medical device at the desired location, such as a fetal trachea. An implant accessory may be a tube, for example, which is operably connected to the medical device. The implant accessory, such as a tube, may be directly and/or indirectly manipulated by the user, such as a surgeon, such as via a separate device connected to the implant accessory external or outside of the living subject. The implant accessory may be a catheter for example. For example, an implant accessory may be a delivery catheter. A “delivery catheter” includes catheters that extend into a fetal trachea via endoscopic channel.

The term “therapeutic balloon” refers to a balloon or bulb that is expandable or inflatable and that is capable of performing a therapeutic activity or function in a living subject (or, in vivo), such as, but not necessarily limited to, occlusion. A balloon includes any type of miniature inflatable balloon that will retain its shape after fluid injection or insufflation. Optionally, a therapeutic balloon disclosed herein is capable of retaining its expanded shape, after fluid insufflation, due to the pressure exerted by the fluid on the internal surfaces (e.g., internal wall) of the balloon, without necessarily requiring rigid or semi-rigid structural element(s) to support the expanded shape. Optionally, the expanded or inflated shape of the balloon corresponds to its therapeutic state, or therapeutic state of a medical device having said therapeutic balloon. In aspects, a therapeutic balloon comprises an expandable hollow body having wall(s) surrounding at least a primary lumen, void, or cavity. The volume of the primary lumen is increased upon insufflation of fluid therein. In some aspects, a balloon has a proximal end, which may be operably connected to an implant-accessory during implantation, and a distal end opposite the proximal end. In some aspects, the distal end comprises an ultrasound-activatable actuator. The distal end may comprise a distal neck with the actuator operably attached to the distal neck. In some aspects, the proximal end comprises a locking mechanism that provides an operable and fluidic connection between the implant-accessory and the therapeutic balloon, wherein the locking mechanism is further capable of facilitating detaching the implant-accessory and fluidically sealing the therapeutic balloon upon detachment of the implant-accessory. Optionally, a therapeutic balloon has a cylindrical shape in its expanded or inflated state. Optionally, a therapeutic balloon has a generally rounded or spherical shape in its expanded or inflated state. A therapeutic balloon can be delivered to and implanted at a target region or target location within a living subject via an implant-accessory. After implantation, a therapeutic balloon is preferably wireless or not operably connected to a guide wire, catheter, or implant-accessory that is external to the living subject.

The term “operably connected” refers to a configuration of components, wherein an action or reaction of one component affects another component, but in a manner that preserves each component's functionality. For example, a catheter may be operably connected to a therapeutic balloon such that the catheter can be used to (re)position the therapeutic balloon and/or the catheter can be used to inject a fluid into the therapeutic balloon. The operable connection may be by a direct physical contact between components. The connection may be indirect, with one or more other components, features, or elements, such as a valve and/or locking mechanism (e.g., valve, luer lock, and/or septum), that indirectly connect the operably connected components. For example, a catheter may be in operable connection with a therapeutic balloon via a valve that itself is connected to the therapeutic balloon and to the catheter.

The term “detachably connected” refers to a configuration of components, wherein the detachably connected component is capable of being detached from another component, optionally via an intermediate component. For example, a catheter may be detachably connected to a therapeutic balloon such that the catheter can be detached from the balloon, optionally via a valve or other detaching/locking mechanism.

The terms “directly” and “indirectly” describe the actions or physical positions of one component relative to another component. For example, a component that “directly” acts upon or touches another component does so without intervention from an intermediary. In contrast, a component that “indirectly” acts upon or touches another component does so through an intermediary (e.g., a third component).

“In fluid communication” or “fluid communication” refers to the arrangement of two or more components such that a fluid can be transported to, past, through, or from one component to another. As used herein, the term “fluidically connected” is equivalent to and interchangeably with “in fluid communication.” For example, in some embodiments, two objects are fluidically connected with one another if a fluid flow path is provided directly between the two objects. In some embodiments, two objects are in fluid communication with one another if a fluid flow path is provided indirectly between the two objects, such as by including one or more intermediate objects or flow paths between the two objects. In one embodiment, two objects present in a body of fluid are not necessarily in fluid communication with one another unless fluid from the first object is drawn to, past, and/or through the second object, such as along a flow path. For example, a catheter that is operably connected to a therapeutic balloon is furthermore fluidically connected to said balloon if a fluid can be injected (directly or indirectly) from the catheter into the therapeutic balloon.

The term “actuator” refers to a component, of a medical device, that can control, activate, or actuate an aspect or state of the medical device. In aspects, the actuator can be activated or triggered remotely by a remote external stimulus. In aspects, activation of an actuator can activate or initiate a transition of a medical device from one state, such as a therapeutic state of the medical device, to another state, such as an expulsion state of the medical device. In aspects, an actuator can be activated between a closed state and an open state, whereby change in the state of the actuator activates or results in a change of state of the medical device. In aspects, a closed state of an actuator is capable of retaining a fluid in a therapeutic balloon by blocking fluid flow through the actuator. In aspects, an open state of an actuator is capable of draining or release a fluid from a therapeutic balloon by allowing the fluid to pass through or around the actuator. In aspects, an actuator is a valve or an actuator comprises a valve. In aspects, an actuator comprises a fluid-escape conduit and a polymer plug, wherein a position and/or a shape (e.g., temporary shape or permanent shape) of the polymer plug either plugs/blocks the fluid-escape element, corresponding to the actuator's closed state, or does not plug/block the fluid-escape element, corresponding to the actuator's open state. In aspects, an actuator comprises a polymer component having one or more shape memory polymers. In aspects, the polymer component is a polymer plug. In aspects, an actuator can be activated or triggered remotely via ultrasound or focused ultrasound such that said actuator is thus is an ultrasound-activatable or focused-ultrasound activatable actuator. For example, an ultrasound-activatable actuator comprises an ultrasound-absorbing shape memory polymer or a polymer component comprising one or more ultrasound-absorbing shape memory polymers.

The term “therapeutic activity” refers to an activity or function of a medical device or component(s) thereof that has therapeutic value such as treating a disease, pathology, condition, or injury, reducing one or more symptoms of a disease, pathology, condition, or injury, preventing a disease, pathology, condition, or injury, preventing symptoms of a disease, pathology, condition, or injury, or the like.

The term “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to a subject, such as a patient in need of treatment; slowing in the rate of degeneration or decline; making the final point of degeneration less debilitating; improving a subject's physical or mental well-being. The treatment or amelioration of symptoms can be based on objective or subjective parameters; including the results of a physical examination, neuropsychiatric exams, and/or a psychiatric evaluation.

A medical device may be capable of being in one or more states. Generally, a medical device is capable of being in one of a plurality of states at any one time, optionally including transitionary or intermediate states. For example, a medical device may have at least an implantation state, a therapeutic state, and an expulsion state. A “state” of a medical device refers to a combination of a configuration of the device and the components, features, and configuration(s) of the components and features of the device. For example, a device comprises one or more components in one state but not in another state. For example, a medical device may comprise and/or be operably connected to an implant-accessory, such as a microcatheter, in the implantation state of the medical device whereas the same device may be free of the implant-accessory and/or not operably connected to the implant-accessory in a therapeutic state and/or an expulsion state of the said device. For example, a therapeutic balloon of a medical device may comprise a fluid in the balloon's lumen when the device is in its therapeutic state, whereas the same balloon may be free of the fluid or have less of the fluid when the same medical device is in its expulsion state and/or its implantation state. For example, one or more components of a device may be configured differently in one state of the device compared to another state of the device. For example, a therapeutic balloon of a medical device may be configured such that it is expanded or inflated, as a result of being insufflated or filled with a fluid, when the medical device is in its therapeutic state, and the same therapeutic balloon may be configured such that it is contracted or deflated, as a result of being free or substantially free of the a fluid, when the medical device is in its expulsion state. Optionally, a medical device has one or more implantation states, one or more therapeutic state, and/or one or more expulsion states. Optionally, a state is not necessarily static but may be dynamic. For example, an implantation state of a medical device may be characterized as or defined by the medical device comprising or being operably connected to an implant-accessory, wherein the therapeutic balloon may be deflated in a first portion of the implantation state (e.g., first implantation state or a first time portion during persistence/existence of the implantation state), inflated in a third portion of the implantation state (e.g., third implantation state or a third time portion during persistence/existence of the implantation state), and the therapeutic balloon may be dynamically transitioning a plurality of intermediate configuration between deflated and inflated in a second portion of the implantation state (e.g., second implantation state or a second time portion during persistence/existence of the implantation state). In aspects, a medical device is configured to be in an implantation state to facilitate implantation of the device in the living subject, in a therapeutic state to facilitate performance of the therapeutic activity, and subsequently in an expulsion state to facilitate expulsion of the medical device from the living subject. In aspects, a medical device is in the implantation state when being implanted in the living subject, subsequently in the therapeutic state when performing the therapeutic activity in the living subject, and further subsequently in the expulsion state when being expelled from the living subject.

The term “biologically-benign fluid” refers to a fluid that is inactive, inert, benign, or beneficial to a living subject when inside the living subject (or, in vivo). A biologically-benign fluid may be a pharmaceutically acceptable excipient or pharmaceutically acceptable carrier, for example. Whether a fluid is biologically-benign may depend on the region(s) or location(s) (e.g., target implantation region of a living subject) which is, will be, and/or could be exposed to said fluid, such as in the course of a medical or therapeutic treatment utilizing a medical device according to aspects herein. Examples of biologically-benign fluids may include, but are not necessarily limited to, water, normal saline solutions, salt solutions (such as Ringer's solution), alcohols, oils, gelatins, and any combination thereof.

The term “shape memory polymer” (or, “SMP”) refers to a polymer that has the ability to return from a deformed shape (a temporary shape) or state to its original shape (a permanent shape) or state when induced by an external stimulus, such as temperature change. This behavior is also referred to as a shape memory behavior. The permanent shape of a shape memory polymer or of a polymer component (optionally, a composite material) therewith is set, established, or defined upon the formation of chemical or physical crosslinks therein. For example, the crosslinking can occur at a temperature approaching (e.g., within 20%), equal to, or greater than a Tₚₑᵣₘ, or a permanent shape temperature or a crosslinking temperature. For example, a permanent shape can be imparted upon or forced upon a shape memory polymer or a polymer component (optionally, a composite material) therewith when or as crosslinking is occurring therein (at a temperature approaching, near, at, or beyond Tₚₑᵣₘ) by constraining at least a portion of the polymer or material (such as in a mold), applying a force to at least a portion of the polymer or material, and/or applying tension or compression to the polymer or material, or mechanically or otherwise deforming the polymer or material during crosslinking or as crosslinking occurs. A temporary shape of a shape memory polymer or a polymer component (optionally, a composite material) therewith can be set by heating the polymer or material to its transition temperature, Tₜᵣₐₙₛ, which may correspond to the polymer's or material's glass transition temperature, Tg, and/or its melt temperature, Tₘ. As the temperature approaches (e.g., within 20%), is equal to, or is greater than Tₜᵣₐₙₛ, the polymer or material can be deformed, optionally mechanically, to establish or set the temporary shape. As the deformation, corresponding to the temporary shape, is applied to the polymer or material, the deformation is preferably maintained, mechanically or otherwise, as the polymer or material is cooled to below its Tₜᵣₐₙₛ, thereby setting or defining the temporary shape of the polymer or material. Crystallization of ordered domains and/or vitrification (where Tₜᵣₐₙₛ=Tₘ and/or Tg, respectively) serves to hold the polymer in its temporary shape until it is again heated to a temperature approaching, equal to, or greater than Tₜᵣₐₙₛ, in which case, in the absence of the deformation (e.g., mechanical force) which established the temporary shape, the polymer or material will spontaneously return to its permanent shape. This transformation in form is known as the shape memory effect.

As used herein, a “shape” of a material refers to its geometry or physical configuration. In aspects, the term “shape” refers to a material's macroscopic geometry or physical configuration as well as one or more microscopic, nanoscopic, and/or atomic physical characteristics, including but not limited to strain and/or stress. Therefore, as used herein, a “shape change” refers to a change in a material's macroscopic geometry or physical configuration and optionally further includes change in one or more microscopic, nanoscopic, and/or atomic physical characteristics, such as a distribution in stress and/or strain in the material. As merely an illustrative example, if a shape memory polymer or polymer component (optionally, a composite material) therewith is programmed to contract as part of the shape change from temporary shape to permanent shape but is fixed on both ends during the shape change (e.g., as the material is heated to Tₜᵣₐₙₛ, or T_{cm,trans} in case of polymer component (optionally, a composite material)), the shape change may be more subtle or limited to a change in forces within the structure. In various aspects, a shape change is an expansion, a contraction, a twisting, an unraveling, a curling, an unfurling, an opening, a closing, a bending, an unbending, a folding, an unfolding, a straightening, a lengthening, a shortening, a redistribution or change in distribution of stress in the material, a redistribution or change in distribution of strain in the material, or any combination of these. In some aspects, a shape change is not necessarily from a temporary shape to the final/original permanent shape, but instead the shape change is complete or stopped at an intermediate shape (e.g., an intermediate shape between temporary shape and permanent shape before original permanent shape is obtained).

The term “transition temperature” or “Tₜᵣₐₙₛ” refers to a characteristic temperature of the phase or shape change corresponding to a shape memory polymer or a polymer component (optionally, a composite material) having a shape memory polymer, or any portion(s) thereof, undergoing or exhibiting a shape memory effect, a shape change, or otherwise a transformation from its temporary shape to its permanent shape when heated from a lower temperature thereto. In some aspects, a transition temperature of a material corresponds to a glass transition temperature and/or melt transition temperature of the material. In some aspects, a material may have two transition temperatures, a glass transition and a melt transition, wherein only the melt transition lies above room temperature, enabling formation of a temporary shape that is stable at room temperature. For example, in aspects, assuming endotherms are plotted as negative, the transition temperature for the melt transition corresponds to the minimum of the negative peak in the 2nd cycle heating curve in differential scanning calorimetry (DSC) data. In aspects, for determining a Tₜᵣₐₙₛ, when plotting DSC data with the positive exotherm convention, as provided herein, for example, the heating curve upon melting (endothermic) will have a negative peak and Tₘ is defined as the minimum. Use of DSC for determining relevant transition temperature(s), useful for aspects disclosed herein, such as Tₘ, T_{c}, and/or Tg is described by Gabbott, P. in “A Practical Introduction to Differential Scanning calorimetry”, Chapter 1, pp. 1-50, in Principles and Applications of Thermal Analysis, 2008 (DOI: 10.1002/9780470697702.ch1), especially section 1.5.2.3 and 1.5.5, which are incorporated herein by reference in their entireties. A polymer component (optionally, a composite material), having one or more shape memory polymers, optionally further comprising one or more additives, can be characterized by a transition temperature of the polymer component (optionally, a composite material), for which the shorthand used herein is T_{cm,trans}. A crosslinked shape memory polymer that is free of additives disclosed herein, or an “additive-free crosslinked shape memory polymer”, can be characterized by a transition temperature of the additive free crosslinked shape memory polymer, or simply T_{pol,trans}.

The terms “crosslinking temperature” and “T_{crosslink}” refer to a characteristic onset or a lower limit temperature of chemical and/or physical crosslinking in a shape memory polymer or a composite material having a shape memory polymer, or any portion(s) thereof, corresponding to the setting, programming, or establishing of its permanent shape when heated from a lower temperature thereto. As used herein, the term “T_{crosslink}” refers to the lower limit or onset temperature of chemical crosslinking as just defined, whereas “Tₚₑᵣₘ” (or, permanent shape programming temperature) refers to the temperature that is actually used or reached for crosslinking and programming/setting the permanent shape. Thus, Tₚₑᵣₘ is equal to or greater than T_{crosslink}. Tₚₑᵣₘ is not necessarily an intrinsic property of the material, but rather depends on the crosslinker (or, crosslinking moiety, or crosslinking precursor) used, and is chosen depending on practical considerations such as, but not limited to, a reasonable reaction time, temperature limits of the mold used, and avoiding decomposition of the SMP. For example, in aspects, a crosslinking temperature corresponds to a temperature at which a thermal initiator decomposes, causing the crosslinking reaction to occur. In some aspects, T_{crosslink} is not necessarily directly measured, but a valid lower limit for Tₚₑᵣₘ, and thus an estimate of T_{crosslink}, can be estimated. For example, in aspects, an upper limit of Tₚₑᵣₘ or range thereof could be defined as the temperature at which the SMP and/or crosslinking moieties thereof decompose. The crosslinking reaction will occur faster the higher the temperature above the lower limit T_{crosslink}. As such, as used herein, Tₚₑᵣₘ corresponds to the lower limit temperature, or onset temperature, of the crosslinking reaction in a given polymer or material. Once the crosslinking step has been completed, (re)heating to T_{crosslink}, Tₚₑᵣₘ, or higher no longer elicits crosslinking or shape programming effect, since the crosslinker has been completely consumed. Generally, heating to T_{crosslink} or Tₚₑᵣₘ does not trigger any shape change in a crosslinked SMP. The minimum time required at a particular given temperature, being approximately equal to or greater than T_{crosslink}, to complete crosslinking can be estimated by a kinetic study where samples of the material are heated to that particular temperature Tₚₑᵣₘ for varying amounts of time. DSC can be used for such a study (see FIG. 19), for example, where bimodal peaks (Tₘ and T_{c}) indicate incomplete crosslinking. The time at which the peaks stabilize at their final shapes, and no longer change, indicates the minimum crosslinking time at a particular temperature at which crosslinking is performed. A composite material having one or more shape memory polymers and further comprising one or more additives, according to aspects disclosed herein, or a monomeric formulation capable of forming the composite material and comprising crosslinking precursor(s) and one or more additives can be characterized by a minimum/onset/lower limit crosslinking temperature of the composite material, for which the shorthand used herein is T_{cm,crosslink}. An actual temperature used to crosslink and program/set a permanent shape for a composite material having one or more shape memory polymers and further comprising one or more additives, according to aspects disclosed herein, or a monomeric formulation capable of forming the composite material and comprising crosslinking precursor(s) and one or more additives may be referred to herein as the permanent shape programming temperature or T_{cm,perm}. In contrast to a composite material having one or more additives, as disclosed herein, an additive-free shape memory polymer comprising crosslinking precursor(s) or an additive-free monomeric formulation having crosslinking precursor(s) and capable of forming a shape memory polymer can be characterized by a minimum/onset/lower limit crosslinking temperature, or simply T_{pol,crosslink}. An actual temperature used to crosslink and program/set a permanent shape for an additive-free shape memory polymer comprising crosslinking precursor(s) or an additive-free monomeric formulation having crosslinking precursor(s) and capable of forming a shape memory polymer may be referred to herein as the permanent shape programming temperature or T_{pol,perm}. For example, in some aspects, crosslinking and permanent shape programming/setting of a composite with DBzP as crosslinking precursor is performed, in some aspects, at 140° C. (Tₚₑᵣₘ) for 8-12 hours, whereas a practical lower limit of crosslinking temperature (T_{crosslink}) of the formulation/mixture may be 91° C., and, in some aspects, a 10-hour HLT is used as lower limit of crosslinking temperature (T_{crosslink}) which may be 73° C. in the case of DBzP for example.

The term “10-hour half-life temperature” or “10-hour HLT” refers to a temperature at which 50% of the substance, compound, or material, such as an organic peroxide crosslinking precursor, will decompose in 10 hours. This parameter corresponds to an optional estimate of lower limit for estimating crosslinking temperature, T_{crosslink}, because a typical crosslinking reaction may be less efficient in a polymer in practice, since the crosslinker is spread out in the polymer matrix in solid state, compared to the 10-hour HLT measurement for a pure crosslinking precursor (e.g., organic peroxide) dissolved in an organic solvent. Relevant 10-hour HLT values for various compounds and descriptions of measurements thereof are available in “Kirk-Othmer Encyclopedia of Chemical Technology”, chapter “PEROXIDES AND PEROXIDE COMPOUNDS, ORGANIC PEROXIDES” (DOI: 10.1002/0471238961.1518070119011403.a01).

As used herein, the term “ultrasound” is intended to be consistent with the term as used in the field of physics. Generally, ultrasound is sound, sound frequencies, sound waves, or acoustic energy characterized by frequencies greater than approximately 20 kHz. In some aspects, ultrasound is characterized by sound wave frequencies of at least approximately 20 kHz and optionally less than or equal to 5 GHz, optionally less than or equal to 1 GHz, optionally less than or equal to 500 MHz, optionally less than or equal to 400 MHz, optionally less than or equal to 300 MHz, optionally less than or equal to 250 MHz, optionally less than or equal to 200 MHz, optionally less than or equal to 150 MHz, optionally less than or equal to 100 MHz, optionally less than or equal to 50 MHz, optionally less than or equal to 30 MHz, optionally less than or equal to 20 MHz, optionally less than or equal to 19 MHz, optionally less than or equal to 18 MHz, optionally less than or equal to 16 MHz, optionally less than or equal to 15 MHz optionally less than or equal to 14 MHz, optionally less than or equal to 10 MHz, optionally less than or equal to 9 MHz, optionally less than or equal to 5 MHz, optionally less than or equal to 4 MHz, optionally less than or equal to 3 MHz, optionally less than or equal to 1 MHz, optionally less than or equal to 900 kHz, optionally less than or equal to 800 MHz, optionally less than or equal to 700 MHz, optionally less than or equal to 600 MHz, optionally less than or equal to 500 MHz. In certain aspects or embodiments herein, the ultrasound frequency range is that which is relevant and useful to a given or specified application or field of applications (e.g., medical devices). Generally, the terms “ultrasound”, “ultrasound frequencies”, “ultrasound waves”, and “ultrasound energy” may be used interchangeably such as when referring to absorption of ultrasound energy by a material or to a beam of ultrasound energy/waves. In various aspects herein, ultrasound, such as a focused ultrasound beam, used to activate or trigger an actuator or a polymer component thereof, according to aspects herein, is characterized by any frequency or range of frequencies recited herein, such as optionally any frequency or any range of frequencies selected from the range of 20 kHz to 5 GHz, wherein any value and range therebetween inclusively is explicitly contemplated herein, and is characterized by an energy intensity selected from the range of 0.1 W/cm² to 100 W/cm², wherein any value and range therebetween inclusively is explicitly contemplated herein, such as optionally selected from the range of approximately 0.5 W/cm² to approximately 5 W/cm², such as optionally selected from the range of approximately 0.5 W/cm² to approximately 3.5 W/cm², such as optionally selected from the range of approximately (e.g., within 20%) 1 W/cm² to approximately (e.g., within 20%) 3 W/cm². In various aspects herein, an actuator or polymer component thereof is capable of being activated or actuated or triggered (e.g., from open state to closed state or vice versa; e.g., a shape change from temporary shape to permanent shape) by a focused ultrasound beam characterized by any frequency/frequencies and any energy intensity/intensities recited herein.

The term “focused ultrasound” refers to non-ionizing ultrasound, ultrasound frequencies/waves, or ultrasound energy that is directional and focused or confined. Focused ultrasound may focused or confined to a beam of ultrasound waves or ultrasound energy. A focused ultrasound beam may be characterized by a focal volume, focal area, or focal point at which the ultrasound intensity, energy density, power, power density, and/or a flux of the ultrasound beam is maximum. A focused ultrasound beam may be formed with the aid of a transducer. Focused ultrasound may be used for therapeutic techniques such as high-intensity focused ultrasound (HIFU).

The term “ultrasound-attenuation characteristic” refers to an empirically-derived or a computationally determined characteristic of a material that quantitatively describes or defines the ability of the material to attenuate ultrasound energy or ultrasound frequencies. An “ultrasound-absorption characteristic” is an ultrasound-attenuation characteristic. The term “ultrasound-absorption characteristic” refers to an empirically-derived or a computationally determined characteristic of a material that quantitatively describes or defines the ability of the material to absorb ultrasound energy or ultrasound frequencies. Ultrasound-attenuation characteristics include, but are not limited to, an ultrasound attenuation coefficient, an ultrasound absorptivity (e.g., molar absorptivity), an ultrasound absorption coefficient (e.g., mass absorption coefficient), and an ultrasound absorption cross-section. In some aspects, an ultrasound-attenuation characteristic is preferably an ultrasound attenuation coefficient.

The term “size characteristic” refers to a property, or set of properties, of a particle that directly or indirectly relates to a size attribute. According to some aspects, a size characteristic corresponds to an empirically-derived size characteristic of a particle or particles being detected, such as a size characteristic based on, determined by, or corresponding to data from any technique or instrument that may be used to determine a particle size, such as but not limiting to optical microscope, electron microscope (e.g., SEM and TEM), optical attenuation (absorbance, scattering, and/or reflectance), and/or a light scattering technique (e.g., DLS). For example, a size characteristic can correspond to a spherical particle exhibiting similar or substantially same properties, such as aerodynamic, hydrodynamic, optical, and/or electrical properties, as the particle(s) being detected. According to some aspects, a size characteristic corresponds to a physical dimension, such as a cross-sectional size (e.g., length, width, thickness, or diameter).

The terms “substantially” and “approximately” interchangeably refer to a property, condition, or value that is within 20%, 10%, within 5%, within 1%, optionally within 0.1%, or is equal to a reference property, condition, or value, respectively. The term “substantially equal”, “substantially equivalent”, or “substantially unchanged”, when used in conjunction with a reference value (e.g., describing a property or condition), refers to a value that is within 20%, within 10%, optionally within 5%, optionally within 1%, optionally within 0.1%, or optionally is equal to the provided reference value. For example, a temperature is substantially equal to 100° C. (or, “is substantially 100° C.” or “is approximately 100° C.”) if the value of the temperature is within 20%, optionally within 10%, optionally within 5%, optionally within 1%, within 0.1%, or optionally equal to 100° C. The term “substantially greater”, when used in conjunction with a reference value, refers to a value that is at least 1%, optionally at least 5%, optionally at least 10%, or optionally at least 20% greater than the provided reference value. The term “substantially less”, when used in conjunction with a reference value refers to a value that is at least 1%, optionally at least 5%, optionally at least 10%, or optionally at least 20% less than the provided reference value. As used herein, the terms “substantially” and “approximately” are equivalent and interchangeable. As used herein, the term “about” means a range of values including the specified value, which a person of ordinary skill in the art would consider reasonably similar to the specified value. In embodiments, “about” means within a standard deviation using measurements generally acceptable in the art. In embodiments, “about” means a range extending to ±10% of the specified value. In embodiments, “about” means the specified value.

The term “moiety” refers to a group, such as a functional group, of a chemical compound or molecule. A moiety is a collection of atoms that are part of the chemical compound or molecule. Crosslinking moieties disclosed herein include moieties characterized as monovalent, divalent, trivalent, etc. valence states, or any combination thereof. Generally, but not necessarily, a moiety comprises more than one functional group.

The term “NTP” refers to the set of conditions defined as normal temperature and pressure, which are a temperature of 20° C. (293.15 K, 68° F.) and an absolute pressure of 1 atm (14.696 psi, 101.325 kPa).

The term “wt. %” refers to a weight percent, or a mass fraction represented as a percentage by mass. As used herein, a concentration of the first additive in the one or more shape memory polymers may be characterized using a wt. % of the first additive in the one or more shape memory polymers, wherein wt. %=[(weight of first additive)/(weight of the one or more polymers only, excluding first additive)].

In an embodiment, a composition or compound of the invention, such as an alloy or precursor to an alloy, is isolated or substantially purified. In an embodiment, an isolated or purified compound is at least partially isolated or substantially purified as would be understood in the art. In an embodiment, a substantially purified composition, compound or formulation of the invention has a chemical purity of 95%, optionally for some applications 99%, optionally for some applications 99.9%, optionally for some applications 99.99%, and optionally for some applications 99.999% pure.

### DETAILED DESCRIPTION OF THE INVENTION

In the following description, numerous specific details of the devices, device components and methods of the present invention are set forth in order to provide a thorough explanation of the precise nature of the invention. It will be apparent, however, to those of skill in the art that the invention can be practiced without these specific details.

Given the fatal complications that may occur with the second FETO procedure, there is a need in the art for remote deflation of the tracheal balloon without surgical intervention Provided herein are medical devices that address at least the issues described above, such as remote deflation of a tracheal balloon without invasive surgical procedures being necessarily. The medical devices disclosed herein can be used for FETO procedures but without needing the second procedure of having the device or balloon invasively removed, thereby making FETO procedures safer and potentially expanding the eligibility for the procedure to save more lives.

Shape memory polymers (SMPs) are materials which can change shape between a temporary shape and a permanent shape upon exposure to an external stimulus such as heat, light, ultrasound, or an electric or magnetic field [5]. Both the temporary and permanent shapes can be programmed through processing and synthesis of the material. Such materials are characterized by a transition temperature, Tₜᵣₐₙₛ, at which the shape change is triggered due to heating, for example. The shape memory effect, a spontaneous transformation from the temporary to permanent shape, can be reversible or irreversible, and can be triggered directly by the stimulus or indirectly through heat produced by applying the stimulus. SMPs have been widely utilized for a range of medical applications such as self-expanding stents, drug delivery devices, and self-tightening sutures [5]. However, a balloon valve which incorporates the shape memory effect in its opening mechanism and can be activated remotely using an external stimulus such as ultrasound has not been previously realized or demonstrated, to the inventors' knowledge. Current state-of-the-art therapeutic balloons are not remotely deflatable.

The medical devices disclosed herein include a therapeutic balloon, such as a tracheal balloon useful for a FETO procedure, and an actuator that can be remotely activated or actuated using focused ultrasound. In various aspects, the device has a therapeutic state characterized by the therapeutic balloon being inflated, such as to provide the therapeutic activity/function of tracheal occlusion. In various aspects, the actuator is in a closed state during the therapeutic activity, such as tracheal occlusion, in which case a fluid is retained in the balloon thereby keeping the balloon inflated to maintain occlusion. In various aspects, the medical device may remain implanted in a living subject in the therapeutic state, neither directly nor indirectly physical connected to any component, device, or accessory that is external of the living subject, for a prolonged period of time, such as at least several weeks. In various aspects, the actuator can be remotely activated via a focused ultrasound beam, which is non-invasively directed at/into the living subject, which, in various aspects, activates or actuates the open state of the actuator, thereby allowing fluid to drain out of the therapeutic balloon and further thereby deflating the balloon, which corresponds to the expulsion state of the device. In various aspects, once the therapeutic balloon is deflated, and the medical device is therefore in its expulsion state, the therapeutic balloon no longer provides occlusion and may be expelled naturally by the body or natural bodily process(es) of the living subject.

Various aspects disclosed herein relate to remote deflation of a detachable therapeutic balloon positioned within the human fetal trachea and placed at an earlier time using endoscopic technique. In various aspects, the endoscopic detachable balloon is equipped with a valve plug made of a shape memory polymer material that changes shape upon exposure to an external stimulus, which is focused ultrasound in a preferred embodiment. In various aspects, the change in shape of the valve plug upon activation with the external stimulus opens the valve, allowing the balloon fluid to escape and causing balloon deflation.

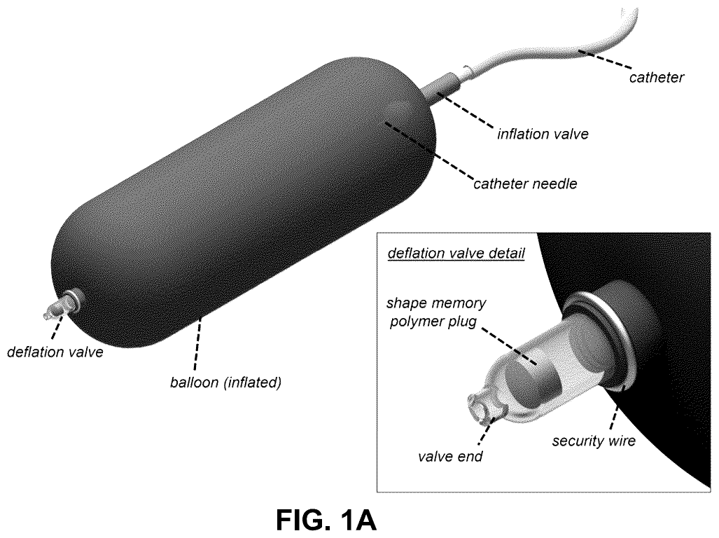

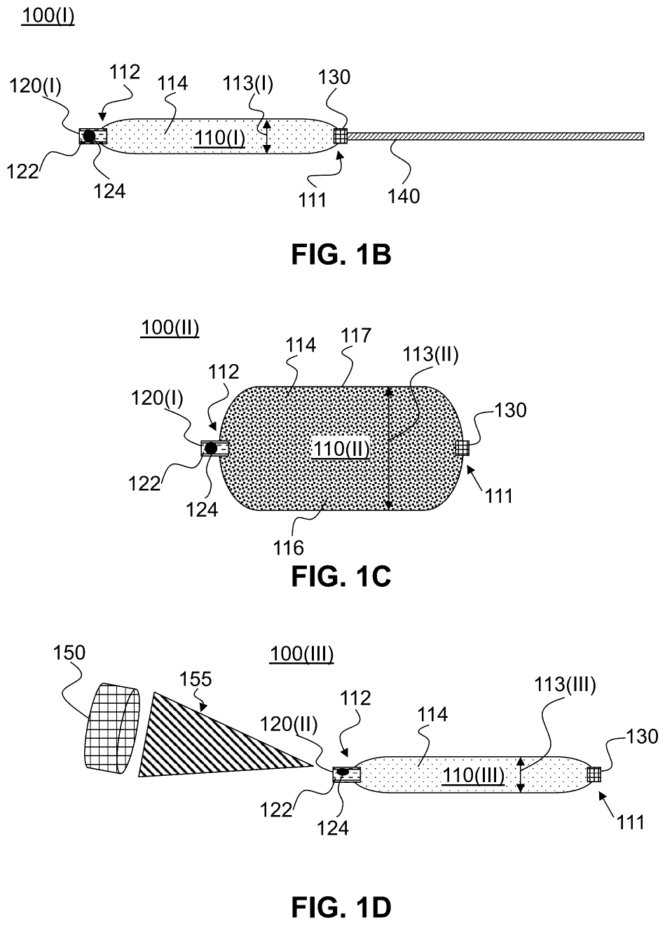

In various aspects, an endoscopic detachable balloon is outfitted with two biocompatible valve plugs on each end of an inflatable balloon (FIG. 1). In various aspects, one of the plug valves accommodates an access needle which is attached to the delivery catheter. In various aspects, the delivery catheter with the needle tip is used to inflate the balloon with fluid once the balloon is positioned at the desired target location. In various aspects, the contralateral valve plug is made from a shape memory polymer. In various aspects, the shape memory polymer has a permanent shape and a temporary shape that are programmed during device fabrication. In various aspects, upon exposure to the external stimulus, the polymer transforms from its temporary shape to its permanent shape, opening a channel for the balloon fluid to escape and deflate the balloon. In various aspects, Tₜᵣₐₙₛ is higher than the physiologic temperature and thus the valve will not be activated by normal body temperature fluctuations.

In various aspects, focused ultrasound is used as the external stimulus to achieve shape change.

In various aspects, once the polymer has undergone a shape change allowing the fluid within the balloon to escape, the balloon will be ejected out of the trachea by the fluid pressure that has built up in the distal trachea. In addition, at the time of 34 weeks gestation, the fetus will be in a head down position pointed toward the mother's pelvis. In various aspects, gravity will cause the balloon to exit the trachea.

In various aspects, when the valve plug has changed shape, it does not detach itself from the balloon. In various aspects, it remains part of the balloon device so that all components are removed together.

Some aspects herein include a delivery catheter. In various aspects, a delivery catheter is sufficiently thin that it will go through the endoscopic channel. In various aspects, positioned at one end of the catheter is a very thin metal needle that is used to connect and access the inner chamber of the balloon. In various aspects, the opposite end of the catheter has a luer lock port which allows fluid injection to fill the balloon.

In various aspects, once the balloon has been positioned within the mid-trachea, it is inflated using the delivery catheter. In various aspects, the friction force applied to the trachea by the balloon holds the balloon in place. In various aspects, steady pulling of the delivery catheter allows the needle to detach from the balloon valve component.

In various aspects, the balloon component can be made of silicone, latex or another biocompatible material that can be inflated and holds the fluid within the balloon without leakage for a prolonged period.

In various aspects, the fluid used to inflate the balloon can be sterile water or another biocompatible fluid that enhances ultrasound visualization or magnetic resonance imaging (MRI) visualization of the balloon. In various aspects, the fluid used may contain a therapeutic compound or compounds that is released upon valve opening.

In various aspects, the polymer used in the valve plug is biocompatible. In various aspects, it can also be coated with a biocompatible material such as silicone to prevent contact with the amniotic fluid.

In various aspects, the detached balloon that has been deflated and expelled out from the trachea does not need to be removed from the uterus. In various aspects, if the fetus swallows the balloon accidentally, it will be expelled through the anus along with meconium stool after birth. In various aspects, the detachable balloon are radio-opaque and can be detected with an x-ray study if there is a need to look for the balloon.

In various aspects, the materials used to make the detachable balloon are compatible with a magnetic resonance imaging (MRI) scanner.

Some aspects herein are related to a method of manufacturing an ultrasound controlled remotely deflatable endoscopic detachable balloon. In various aspects, a method includes constructing a balloon with means to inflate and deflate remotely and a delivery system to deploy the detachable balloon to the target location.

In various aspects, a method can include the use of an endoscope to visualize the fetal trachea and to insert the wireless detachable balloon that can respond to an external stimulus to deflate and release itself from the tracheal lumen.

In various aspects, a method can include how the insufflation valve and the heat sensitive polymer valve can be aligned as a single unit or separated by the balloon component. In various aspects, the balloon component can be connected to more than one polymer valve to work as a back-up system in case the primary remotely controlled valve fails to respond and does not allow balloon deflation.

In various aspects, a long-lasting, wireless, biocompatible detachable balloon is integrated with insufflation valve and deflation valve. In various aspects, once endoscopically deployed within a human fetal trachea, it occludes the trachea preventing pulmonary fluid to exit the lung. In various aspects, the fluid buildup causes the lung to expand and stimulate growth. In various aspects, after adequate period of lung growth, the balloon is deflated remotely without physical contact with the balloon. In various aspects, the balloon is equipped with a shape memory polymer plug that changes its shape upon exposure to an external stimulus. In various aspects, the balloon which was occluding the fetal trachea is deflated upon application of the external stimulus and expelled from the trachea. With the trachea open, normal lung development can proceed.

In various aspects, an implantation system for long-term fetal tracheal occlusion is disclosed. In various aspects, the detachable balloon can fit inside a fetal trachea to occlude the lumen and prevent pulmonary fluid from exiting the lung.

In various aspects, an endoscopically implanted detachable balloon can be remotely controlled to deflate at a designated time without a surgical intervention. In various aspects, the an ultrasound probe placed on the mother's abdomen is able to detect the tracheal balloon. In various aspects, a secondary probe with focused ultrasound is used to activate the polymer valve with positioning guidance from the diagnostic ultrasound. In various aspects, a shape memory effect is triggered, causing fluid to exit the balloon and the balloon to deflate. The shape memory polymer used for the valve plug will absorb ultrasound more strongly than adjacent tissues or nearby bone, leading to selective heating of the plug without exposure of surrounding tissues to dangerous temperatures.

In various aspects, the valve plug is constructed from a semicrystalline shape memory polymer that is either a crosslinked elastomer or a block copolymer. The crosslinks may be chemical or physical. The crosslinker used varies depending on the choice of polymer and polymerization method. In various aspects, the plug may contain an additive that is incorporated into the polymer matrix to enhance the heating response of the material upon exposure to the external stimulus. In various aspects, the additive may also serve to enhance the diagnostic ultrasound imaging signal to detect and locate the valve plug.

In various aspects, the valve plug is constructed from a chemically crosslinked elastomer.

In various aspects, the detachable balloon can be packaged using materials that are biocompatible and is stable within the fetal trachea for many weeks or the duration needed for pulmonary growth stimulated by tracheal occlusion.

In various aspects, the remotely controlled detachable balloon has no electronic components, battery, or other intrinsic power source to accentuate the valve component. In various aspects, it does not need physical manipulation to deflate the balloon.

In various aspects, the detachable balloon is magnetic resonance imaging (MRI) safe since it has no ferromagnetic component present in excess of published safety limits.

An implantable, detachable, and removable medical device 100, according to various aspects disclosed herein, is shown in FIG. 1B in an implantation state 100(1), according to aspects herein. Medical device 100 comprises a therapeutic balloon 110 and a focused-ultrasound activatable actuator 120. Therapeutic balloon 110 comprises a proximal end 111 and a distal end 112. Actuator 120 is optionally at the distal end 112 of therapeutic balloon 110. Medical device 100 optionally comprises a locking mechanism 130, optionally at proximal end 111 of therapeutic balloon 110. Therapeutic balloon 110 further comprises a lumen 114, or an internal void or cavity.

Optionally, the device in its implantation state 110(1) comprises therapeutic balloon 110 being in its deflated/contracted state 110(1), such as to facilitate it being maneuvered in vivo within a living subject to a target region. Therapeutic balloon in its deflated state 110(1) has a cross-sectional diameter 113(1). Optionally, therapeutic balloon in its deflated state 110(1) is free or substantially free of a fluid, such as a biologically-benign fluid.

The device in its implantation state 100(1) comprises an implant-accessory 140 operably attached thereto, such as operably attached to the therapeutic balloon 110(1), such as optionally operably and fluidically attached to locking mechanism 130. Locking mechanism 130 is capable of attaching to implant-accessory 140 and is further capable of having a fluid pass through it between implant accessory 140 and therapeutic balloon 110 when implant accessory 140 is attached thereto. Locking mechanism 130 is also preferably capable being fluidically sealed or fluidically isolating a fluid inside therapeutic balloon 110 from fluids external to device 100. Optionally, in some aspects, locking mechanism 130 comprises a Luer lock mechanism. Optionally, in some aspects, locking mechanism 130 comprises a septum for providing fluidic isolation while also allowing for fluidic communication between implant accessory 140 and lumen 114, such as via an optional needle of implant accessory 140 (e.g., a catheter with needle). Optionally, locking mechanism 130 is a valve (e.g., “inflation valve”).

Implant-accessory 140 is an accessory, component, feature, or tool that facilitates implantation of the medical device in a desired region or location of the living subject. For example, implant accessory 140 may allow for a user, such as a surgeon, to manipulate the positioning or otherwise move the medical device within the living subject from one location to another in order to ultimately position the medical device at the desired location, such as a fetal trachea. In aspects, implant accessory 140 is a tube or catheter, such as a microcatheter.

Actuator 120 optionally comprises a fluid-escape conduit 122. Actuator 120 optionally comprises a polymer component 124 that is (focused-)ultrasound-activatable, such that polymer component 124 or one or more portions thereof are capable of undergoing a shape change upon exposure to (focused) ultrasound. In aspects, polymer component 124 comprises one or more shape change polymers (SMPs), optionally further comprising one or more additives, the shape change of which, such as from a temporary shape to a permanent shape, can be activated or triggered via (focused) ultrasound. Fluid-escape conduit 122 is a fluid flow channel through which a fluid may pass, such as from lumen 114, when actuator 120 is in its open state 120(11). Optionally, the medical device in its implantation state 110(1) comprises actuator 120 being in its closed state 120(1), which may prevent a fluid from escaping or leaking out of distal end 112 of therapeutic balloon 110. Polymer component 124 is optionally a plug. In some aspects, actuator 120 being in its closed state 120(1) is characterized by polymer component 124 having a temporary shape configured to plug or block fluid-escape conduit 122 such that a fluid may not flow past polymer component 124. As such, actuator 120 may behave as a valve. For example, polymer component 124 may have a temporary shape, such as a cylinder or disk, whose cross-sectional diameter is at least equal to or larger than a diameter of the fluid-escape conduit 122.

FIG. 1C shows an implantable, detachable, and removable medical device in its therapeutic state 100(11), according to various aspects disclosed herein. The medical device in its therapeutic state 110(11) is optionally detached from or not operably connected to implant-accessory 140 or any other accessory or device that is at least partially external to the living subject. The medical device in its therapeutic state 110(11) is preferably implanted or provided in a target region of the living subject, such as a human fetal trachea, where a therapeutic activity, such as tracheal occlusion, is desired. The medical device in its therapeutic state 110(11) comprises therapeutic balloon 110 being in its inflated or expanded state 110(11), as shown in FIG. 1C. The therapeutic balloon in its inflated state 110(11) or lumen 114 thereof is optionally filled with a fluid 116, such as a biologically-benign fluid, such as a saline solution. When the therapeutic balloon in its inflated state 110(11), locking mechanism 130 is preferably providing a fluidic seal or fluidic isolation to prevent leakage of fluid 116 out of the therapeutic balloon. Actuator 120 is also preferably in its closed state 120(1) when therapeutic balloon is in its inflated state 110(11) to prevent leakage or loss of fluid 116 out of the therapeutic balloon. The therapeutic balloon in its inflated state 110(11) is characterized by a cross-sectional diameter 113(11), which is greater than 113(1). Optionally, the therapeutic balloon is maintained in its inflated state 110(11) solely by pressure imparted on the walls 117 of the balloon by fluid 116 inside lumen 114.

Between the device as shown in FIG. 1B and the device as shown in FIG. 1C, a fluid 116, such as a biologically-benign fluid, may be provided to or injected into balloon 110 or lumen 114 thereof, optionally via implant-accessory 140, which may be a catheter, connected to locking mechanism 130 whereby the fluid may flow from implant-accessory 140 through locking mechanism 130 to lumen 114 of balloon 110. Optionally, therefore, device 100 may be characterized as also comprising an intermediate or secondary implantation state wherein the device is in its target region, such as a trachea, and implant-accessory 140 is operably connected thereto and fluid is being injected into balloon 110 via said implant-accessory 140. After a desired amount of fluid 116 is injected, the amount of fluid being selected to inflate the balloon to a desired volume or cross-sectional diameter 113(11), for example, to achieve the desired therapeutic activity, such as occlusion of a vessel, such as tracheal occlusion.

FIG. 1D shows an implantable, detachable, and removable medical device in its expulsion state 100(111), according to various aspects disclosed herein. In aspects, activation of the device's expulsion state 100(111) is triggered or activated by exposure of actuator 120 to a focused ultrasound beam 155, which is generated and directed remotely at the device or actuator thereof via an ultrasound-generation device 150, which may comprise a transducer, which is preferably located external to or outside of the living subject in which medical device 100 is implanted, such that there is no direct physical connection between medical 100 and ultrasound generation device 150 or any physical component thereof. The medical device in its expulsion state 110(111) is characterized by therapeutic balloon 110 being in its deflated or contracted state, which is denoted in FIG. 1D as 110(111) to illustrate that the therapeutic balloon is not necessarily deflated equally or identically to its state during implantation. Likewise, a cross-section diameter of the therapeutic balloon, when the medical device is in its expulsion state 110(111), is indicated as 113(111) to illustrate that said diameter is not necessarily equal to the cross-sectional diameter 113(1) when the device is being implanted. For example, therapeutic balloon in its expulsion state 110(111) may be free of fluid 116 or may have some residual amount of fluid 116 such that there is not sufficient fluid 116 to inflate the balloon. In various aspects, medical device in its expulsion state 100(111) is characterized by actuator 120 being in its open state 120(11). In aspects, actuator 120 is actuated/activated/triggered from its closed state to its open state by its exposure to ultrasound beam 155. In various aspects, actuator 120 is opened as a result of polymer component 124 undergoing a shape change from a temporary shape to a permanent shape as a result of the polymer component being exposure to ultrasound beam 155. Optionally, for example, a cross-sectional dimension (e.g., diameter) of polymer component 124 shrinks to a size less than a cross-section dimension (e.g., diameter) of fluid-escape conduit 122, thereby allowing fluid 116 to escape from balloon 110 or lumen 114 thereof into the surroundings. For example, in some aspects, a temporary shape of polymer component 124 is a disk with a larger diameter than that of fluid-escape conduit 122, thereby plugging conduit 122; for example, in some aspects, a permanent shape of polymer component 124 is a cylinder with a smaller diameter than that of fluid-escape conduit 122, thereby unplugging or opening conduit 122 for fluid flow.

In aspects, with the balloon being deflated when the device is in its expulsion state, the device no longer occludes a vessel, such as a trachea, and may be expelled with a bodily fluid(s) or bodily waste via a natural bodily process(es) of the living subject. The device may then be recovered once it is fully expelled from the living subject by the living subject.

In some aspects, it is preferable for polymer component 124 to remain attached or connected or otherwise a part of device 100 during the entirety of expulsion state 100(111), until at least the time when the device is completely expelled or removed from the living subject. This may be achieved by a variety of ways, and aspects herein are not limited by any particular approach. For example, in an aspect, actuator 120 may comprise a taper or reduced diameter at its distal end (e.g., the end farthest from the balloon) such that the taper or reduced diameter is less than one or more dimensions of the permanent shape of polymer component 124, thereby physically preventing it from escaping. For example, in an aspect, polymer component 124 may be physically and/or chemically (e.g., adhesive) attached to actuator 120 or conduit 122 thereof to prevent release of polymer component 124 into the living subject.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

### Example 1: Additional Discussion of Exemplary Applications

A long-lasting, wireless, biocompatible detachable balloon integrated with insufflation valve and deflation valve is constructed for the purpose of treating fetuses with congenital diaphragmatic hernia. Once endoscopically deployed within a human fetal trachea, it occludes the trachea preventing pulmonary fluid to exit the lung. The fluid buildup causes the lung to expand and stimulate growth. After adequate period of lung growth, the balloon is deflated remotely without physical contact with the balloon. The balloon is equipped with a shape memory polymer plug that changes its shape upon exposure to an ultrasound stimulus. The balloon which was occluding the fetal trachea is deflated upon application of the ultrasound activation and expelled from the trachea. With the trachea open, normal fetal lung development can proceed.

Congenital diaphragmatic hernia (CDH) affects approximately 1 in 4000 births. About 50% of infants with severe CDH die at birth or thereafter. CDH ranks among the most costly and lethal congenital malformation. Excluding anencephaly, trisomy 13 and 18, CDH has the highest risk of neonatal death. Estimated inpatient care of CDH is $400,000 per case with a US annual cost exceeding $350 million. Recent data show that earlier fetal treatment increases the chance of survival for patients with severe CDH and lowers the cost of care.

Congenital diaphragmatic hernia (CDH) is a neonatal disease characterized by incomplete development of diaphragm, causing abdominal content such as the intestine, spleen, stomach, and liver to enter the chest cavity [1]. Space occupation of the chest with abdominal viscera prevents proper development of both ipsilateral and contralateral lung. Neonates born with severe hypoplastic lung due to congenital diaphragmatic hernia will die from inadequate oxygenation upon birth without medical and surgical interventions. Congenital diaphragmatic hernia can be due to genetic disorder but often occurs without any identifiable genetic cause [2].

The current standard treatment for congenital diaphragmatic hernia is to provide adequate oxygenation and ventilation to the newborn upon delivery [1]. Airway control with endotracheal tube intubation is needed if the newborn's respiratory need is severe. If conventional oxygenation support is inadequate, extracorporeal membrane oxygenation (ECMO) may be used. Surgical intervention to reduce the abdominal content from the chest into the abdominal domain and to repair the diaphragm hernia is undertaken once the neonate is hemodynamically stable. Ultimate survival of the neonate is dependent on the degree of hypoplasia or lack of lung development during fetal development. During prenatal surveillance, if the fetus is found to have extremely poor lung development and the chances of survival upon birth are predicted to be extremely low, fetal intervention is offered [3]. Fetoscopic endoluminal tracheal occlusion (FETO) is a surgical method utilized to promote fetal lung growth [4]. During fetal development, pulmonary fluid from the lung passes through the trachea and is expelled into the amniotic space. By occluding the trachea, the pulmonary fluid is forced to accumulate within the trachea causing back pressure into the lung tissue. This increased pressure stimulates lung growth during the period of tracheal occlusion. Two FETO procedures are done; first, tracheal occlusion is established, and second, the trachea is reopened later. The first procedure is done at 27-week gestation. An incision is made on the mother's abdomen. Through the incision, an access port is placed into the uterus. An endoscope is guided through the port, into the mouth of the fetus, then into the fetal trachea. Through the channel within the endoscope, an endoscopic detachable balloon catheter is passed into the trachea. The detachable balloon is positioned within the mid-trachea under endoscopic vision. After fluid inflation of the balloon, the delivery catheter is detached from the balloon. The scope and the catheter are both removed from the uterus. A second procedure to deflate the balloon is done at 34-week gestation. The deflation of the balloon is accomplished by passing a needle through the mother's abdomen and into the uterus under ultrasound guidance. If the tracheal balloon can be visualized with ultrasound probe and the trajectory of the needle path is safe, the balloon can be punctured with the needle. Upon balloon deflation, it is expelled from the trachea by accumulated lung pressure. If the fetal position does not allow needle deflation of the balloon, endoscopic maneuver is done like the first procedure. The balloon is endoscopically punctured and removed from the trachea. Once the baby is delivered at full term, standard medical and surgical treatments are rendered.

FETO procedure complications can be divided into intra-operative and post-procedure complications. Surgical complications mainly occur at the time of second removal procedure. Examples of intraoperative complications include bleeding from accidental injury to the placenta causing fetal demise and difficulty with tracheal balloon deflation. Post-procedure complications include initiation of preterm labor and inadvertent premature delivery. Given the fatal complications and premature delivery that may occur with the second FETO procedure, there is a need for remote deflation and removal of the tracheal balloon without a second surgical intervention.

Aspects herein solve the difficult problem of constructing a detachable endoscopic balloon that responds to ultrasound, deflates and expels from trachea without surgical intervention, thus, saving the fetus with congenital diaphragmatic hernia from the complications of the second fetal surgery. In various aspects, the balloon does not have any electronic component or battery and will be miniaturized to fit into a fetal trachea.

Endoscopically implanted detachable balloon can be remotely controlled to deflate at a designated time without a surgical intervention. An ultrasound probe placed on the mother's abdomen will be able to detect the tracheal balloon. A secondary probe with focused ultrasound will be used to activate the polymer valve with positioning guidance from the diagnostic ultrasound. A shape memory effect will be triggered, causing fluid to exit the balloon and the balloon to deflate. The shape memory polymer used for the valve plug will absorb ultrasound more strongly than adjacent tissues or nearby bone, leading to selective heating of the plug without exposure of surrounding tissues to high temperatures.

An endoscopic detachable balloon is outfitted with two biocompatible valve plugs on each end of an inflatable balloon. One of the plug valves accommodates an access needle which is attached to the delivery catheter. The delivery catheter with the needle tip is used to inflate the balloon with fluid once the balloon is positioned at the desired target location. The contralateral valve plug is made from a shape memory polymer. The shape memory polymer has a permanent shape and a temporary shape that are programmed during device fabrication. Upon exposure to the external stimulus, the polymer transforms from its temporary shape to its permanent shape, opening a channel for the balloon fluid to escape and deflate the balloon (FIG. 5). Since Tₜᵣₐₙₛ is higher than the physiologic temperature, the valve will not be activated by normal body temperature fluctuations. Once the polymer has undergone a shape change allowing the fluid within the balloon to escape, the balloon will be ejected out of the trachea by the fluid pressure that has built up in the distal trachea. In addition, at the time of 34 weeks gestation, the fetus will be in a head down position pointed toward the mother's pelvis. Gravity will cause the balloon to exit the trachea.

The delivery catheter is sufficiently thin that it will go through the endoscopic channel. Positioned at one end of the catheter is a very thin metal needle that is used to connect and access the inner chamber of the balloon. The opposite end of the catheter has a luer lock port which allows fluid injection to fill the balloon. Once the balloon has been positioned within the mid-trachea, it is inflated using the delivery catheter. The friction force applied to the trachea by the balloon holds the balloon in place. Steady pulling of the delivery catheter allows the needle to detach from the balloon valve component.

The balloon component can be made of silicone, latex or another biocompatible material that can be inflated and holds the fluid within the balloon without leakage for a prolonged period. The fluid used to inflate the balloon can be sterile water or another biocompatible fluid that enhances ultrasound visualization of the balloon. The fluid used may contain a therapeutic compound or compounds that is released upon valve opening. The polymer used in the valve plug will be biocompatible. It can also be coated with a biocompatible material such as silicone to prevent contact with the amniotic fluid. The detached balloon that has been deflated and expelled out from the trachea does not need to be removed from the uterus. If the fetus swallows the balloon accidentally, it will be expelled through the anus along with meconium stool after birth. The detachable balloon will be radio-opaque and can be detected with an x-ray study or MRI if there is a need to look for the balloon. The materials used to make the detachable balloon will be compatible with a magnetic resonance imaging scanner.

The impact of the device on future FETO procedures is profound. Presently, the FETO procedure is done only on fetuses who have an extremely low chance of survival. Without FETO, the survival rate for these neonates is 15%. With FETO, survival rate increases close to 50%. In various aspects, medical devices and associated methods disclosed herein may decrease the number of surgical procedures needed by half and prevent complications that may cause the fetus to die. The ability to control balloon deflation remotely allows the surgeons to consider treating moderately severe CDH patients who are normally not considered for treatment.

### Examples 2-6

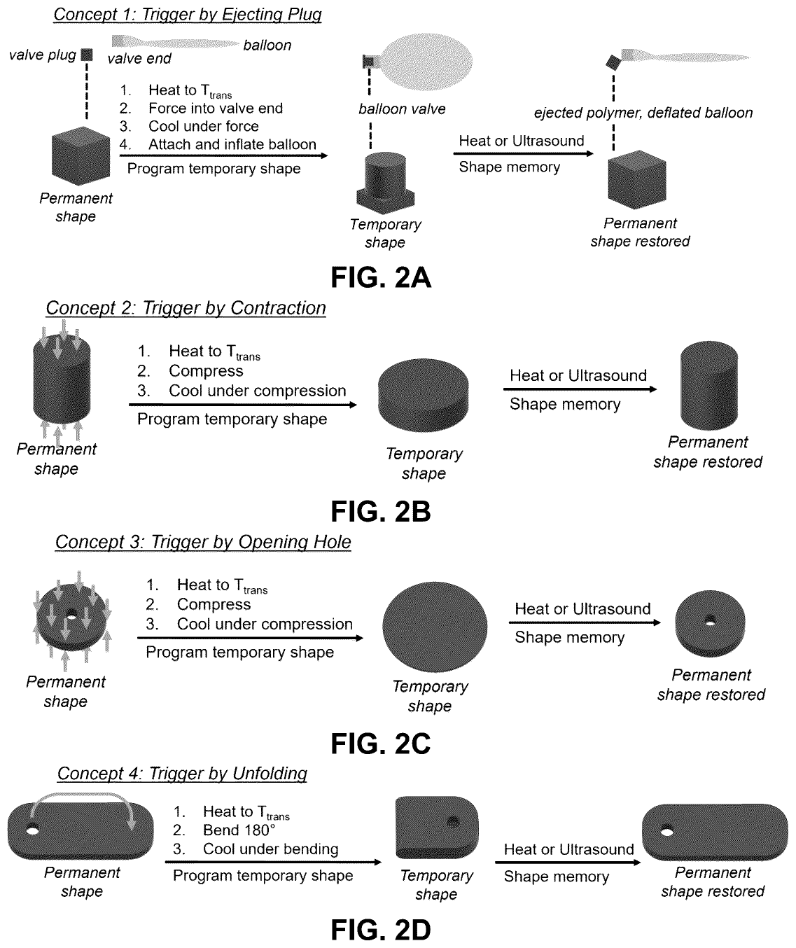

Several valve concepts are fabricated that depend on differing shape memory actuations (FIGS. 2A-2D). First, in Valve Concept 1, a plug that has been forced into a cylindrical valve end while at Tₜᵣₐₙₛ is later ejected from the valve via shape memory induced expansion (FIG. 2A). Secondly, in Valve Concept 2, a tall cylindrical plug is compressed to a temporary shape with larger diameter (FIG. 2B). In this state, it is friction fit into a cyclindrical valve end. Upon ultrasound or heat induced shape memory, the cylinder contracts, decreasing its diameter and opening the valve. Thirdly, in Valve Concept 3, a hole is programmed to open via shape memory (FIG. 2C). Compression to a thin plate forces the hole in the material to close, forming a flat membrane. This membrane is used to seal the valve end. Upon shape memory, the hole reopens, opening the valve. Finally, in Valve Concept 4, a hole is programmed to be uncovered via shape memory induced unfolding (FIG. 2D). The plug is mounted on the valve end with the hole facing the valve interior. Only partial unfolding is required to open the valve under this design. These designs represent certain aspects of an actuator disclosed herein.

### Example 3: Fabrication and Heat Induced Opening of Valve Concept 1

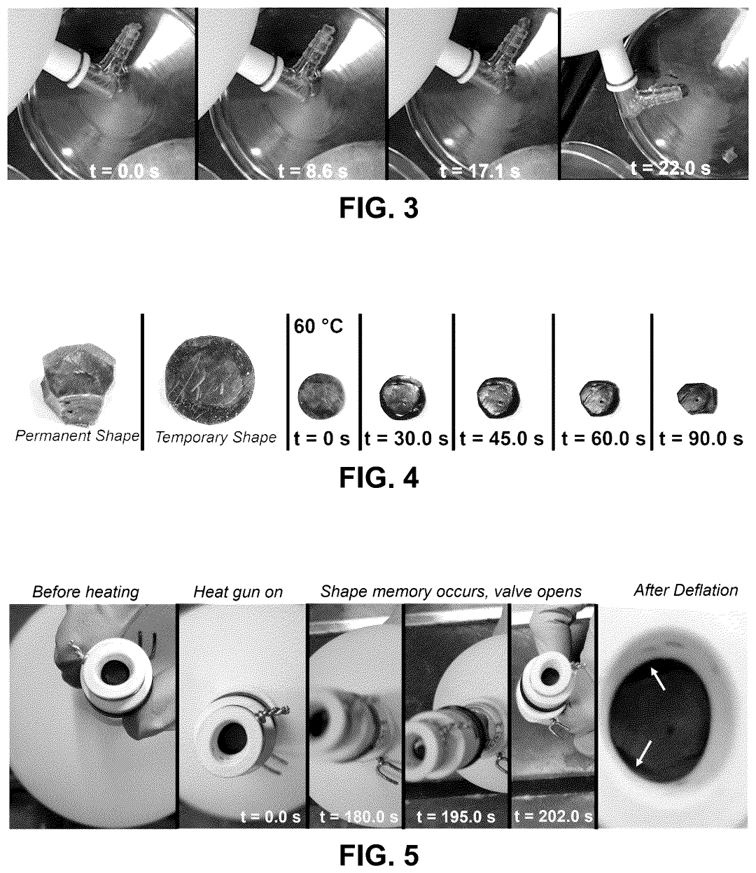

A proof-of-concept valve is constructed from a shape memory polymer, crosslinked poly(cis-cyclooctene) without an additive, as follows. The polymer is cut with a straight razor to a roughly cuboid shape of 5.36 mm×4.71 mm×3.60 mm dimensions to make a plug. The polymer plug is placed in a metal bead bath at 60° C. for 5 minutes to heat the material to Tₜᵣₐₙₛ as observed by the sample turning translucent. The hot sample is forced into the end of a glass 14/20 joint adapter which has a cylindrical channel. Upon cooling to room temperature, a balloon is inflated with air and placed onto the other end of the adapter to construct a proof-of-concept valve (FIG. 3). The balloon is secured with a twisted copper wire and the assembled valve is observed to be airtight and stable for 5 minutes. A heat gun is then used to heat the end of the valve, and the polymer is observed to expand as Tₜᵣₐₙₛ is reached. Eventually, the polymer is ejected from the valve and the balloon is observed to deflate fully (FIG. 3). This design may be undesirable in some aspects or application because the plug may become separated from the device. Therefore, a tether can be added to prevent separation of the plug.

### Example 3: Fabrication and Heat Induced Opening of a Shape Memory Plug for Valve Concept 2

A proof-of-concept plug is constructed from a shape memory polymer composite, crosslinked poly(cis-cyclooctene) containing Fe₃O₄ nanoparticles (MK-1-13D), as follows. The nanoparticles are included in the polymer to enhance ultrasound induced heating. The material is cut into a roughly cylindrical shape of diameter 5.74 mm and height 4.98 mm. The material is then heated to Tₜᵣₐₙₛ by placing it in an oven at 130° C. for 5 minutes. While hot, the material is compressed using a poly(methyl methacrylate) screw clamp with an 8 mm diameter mold insert. Cooling to room temperature and then over dry ice for 10 minutes results in a flat, cylindrical sample of 8.14 mm diameter and 1.93 mm height. Allowing the sample to equilibrate at room temperature completes fabrication and shape memory programming of the prototype valve plug. The plug is placed in a glass dish in a metal bead bath set to 60° C. and observed to contract in diameter, restoring the original dimensions (final dimensions 5.90 mm diameter×4.95 mm height, FIG. 4).

The same sample is then recompressed following the above procedure and used to fabricate an actuator, or valve, according to aspects herein. Before use, the sample had dimensions 7.61 mm diameter×2.33 mm height. The sample is placed into the narrow end of a 14/20 rubber septum which had been punched with a 4 mm biopsy punch (Healthlink Biopunch). A copper wire is used to secure the sample, ensuring effective sealing. The septum is attached to a 14/20 glass adapter and wrapped with electrical tape. To the other end of the adapter, a balloon is attached after inflating. The assembly (FIG. 5) is observed to hold air securely for 5 minutes. A heat gun is then aimed at the valve end. Due to the insulating septum, the valve takes some time to heat. After ˜3 minutes, the balloon began to deflate and quickly deflated over ˜20 seconds. The plug is measured afterwards and is found to have undergone shape change with final dimensions 5.74 mm diameter×4.56 mm height. Gaps denoted by arrows in FIG. 5 can be seen between the plug and the inner wall of the septum which allowed the air to escape.

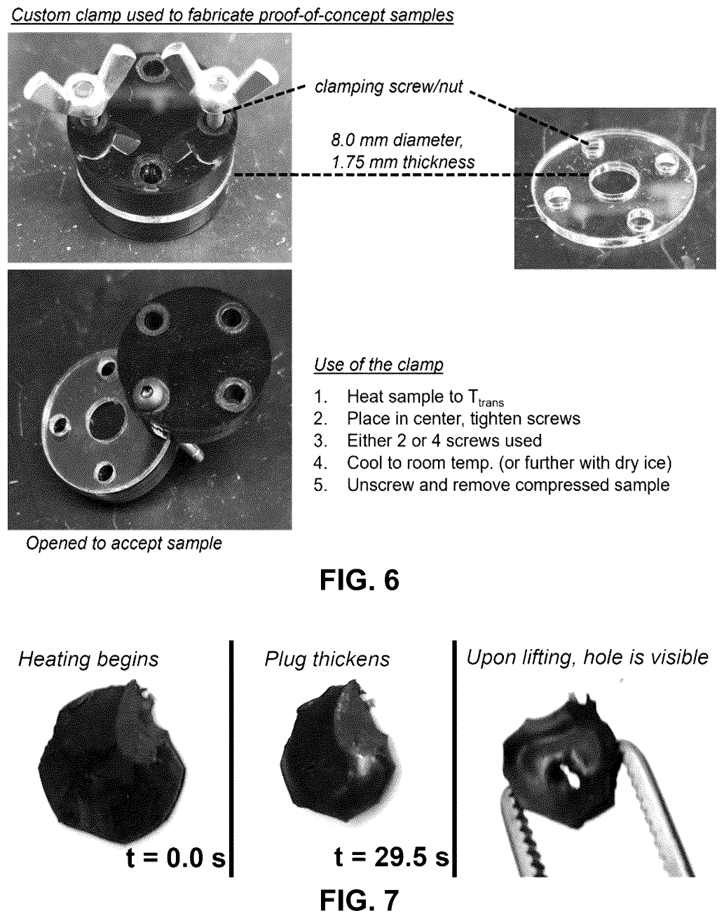

A custom screw clamp is fabricated from poly(methyl methacrylate) by laser cutting and consists of 3 pieces (FIG. 6). The 4 outer holes can accommodate up to 4 M3 screws and wing nuts. The optional interior piece with an 8 mm diameter hole may be used to mold the material to create a 2 mm thickness disc.

### Example 4: Fabrication and Heat Induced Opening of a Shape Memory Plug for Valve Concept 3

A proof-of-concept plug is constructed from a shape memory polymer composite, crosslinked poly(cis-cyclooctene) containing Fe₃O₄ nanoparticles (MK-1-7H), as follows. The material is cut into a rough cylinder of diameter 7.47 mm and thickness 2.80 mm. A hole is punched in the center using a needle of 3.38 mm outer diameter to establish the permanent shape (FIG. 7) which has a hole of 2.08 mm diameter. The material is then heated to Tₜᵣₐₙₛ by placing it in an oven at 130° C. for 5 minutes. While hot, the material is compressed using a poly(methyl methacrylate) screw clamp without the transparent insert piece. Cooling to room temperature completes fabrication and shape memory programming of the prototype valve plug. The plug with this processing does not have a discernable hole. The plug is placed on a hot plate set to 60° C. and observed to expand, reopening the hole (FIG. 7).

### Example 5: Fabrication and Heat Induced Opening of a Shape Memory Plug for Valve Concept 4

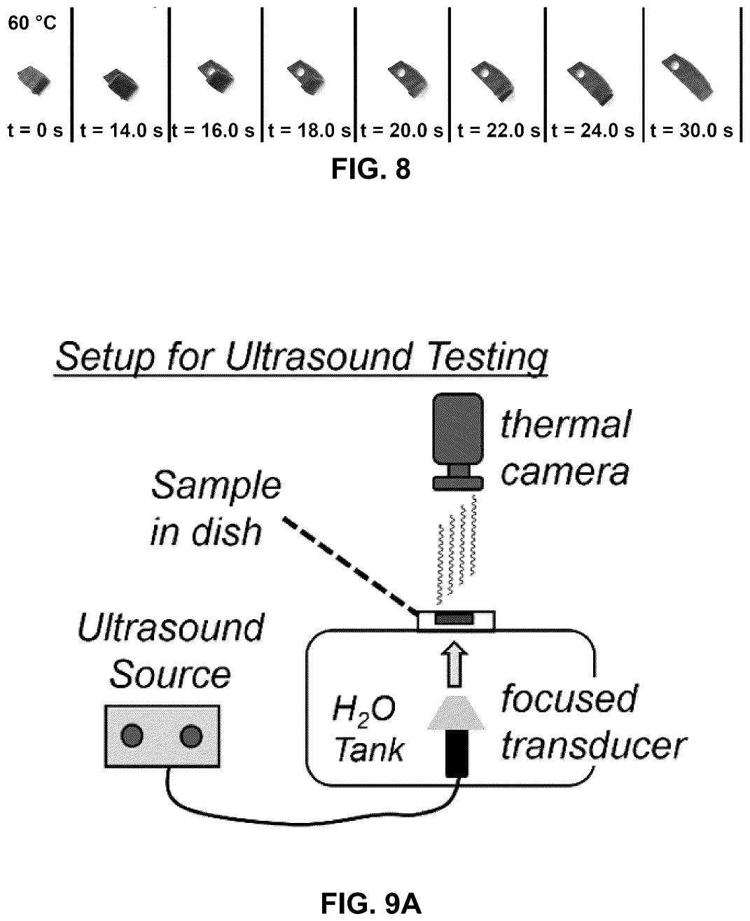

A proof-of-concept plugs constructed from a shape memory polymer composite, crosslinked poly(cis-cyclooctene) containing Fe₃O₄ nanoparticles (MK-1-13E), as follows. The material is cut into a rectangular strip of dimension 9.47 mm×3.19 mm×1.26 mm. A hole is punched in one end using a disposable tissue biopsy punch (1.5 mm, Integra Miltex). The material is then heated to Tₜᵣₐₙₛ by placing it in a metal bead bath at 60° C. for 5 minutes. While hot, the material is folded 180° and held using forceps. Cooling to room temperature completes fabrication and shape memory programming of the prototype valve plug. The plug s placed into a metal bead bath at 60° C. and observed to unfold, revealing the hole (FIG. 8).

### Example 6: Ultrasound Induced Unfolding of a Shape Memory Polymer Plug for Valve Concept 4

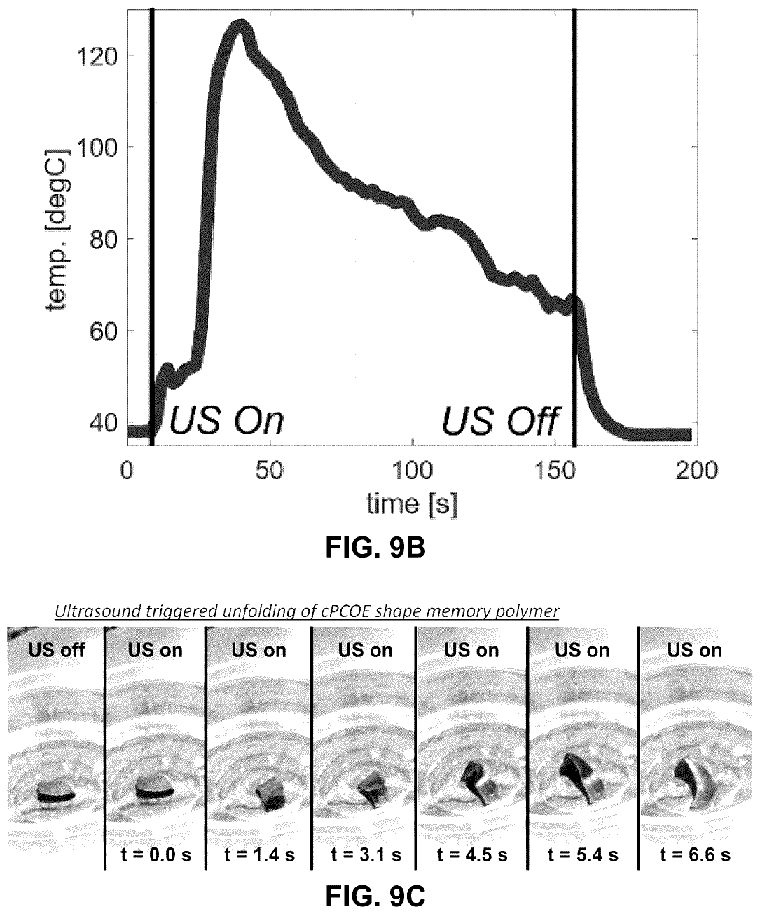

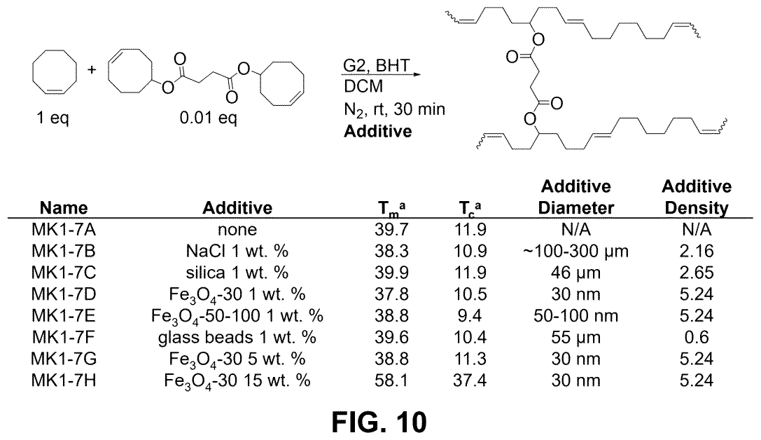

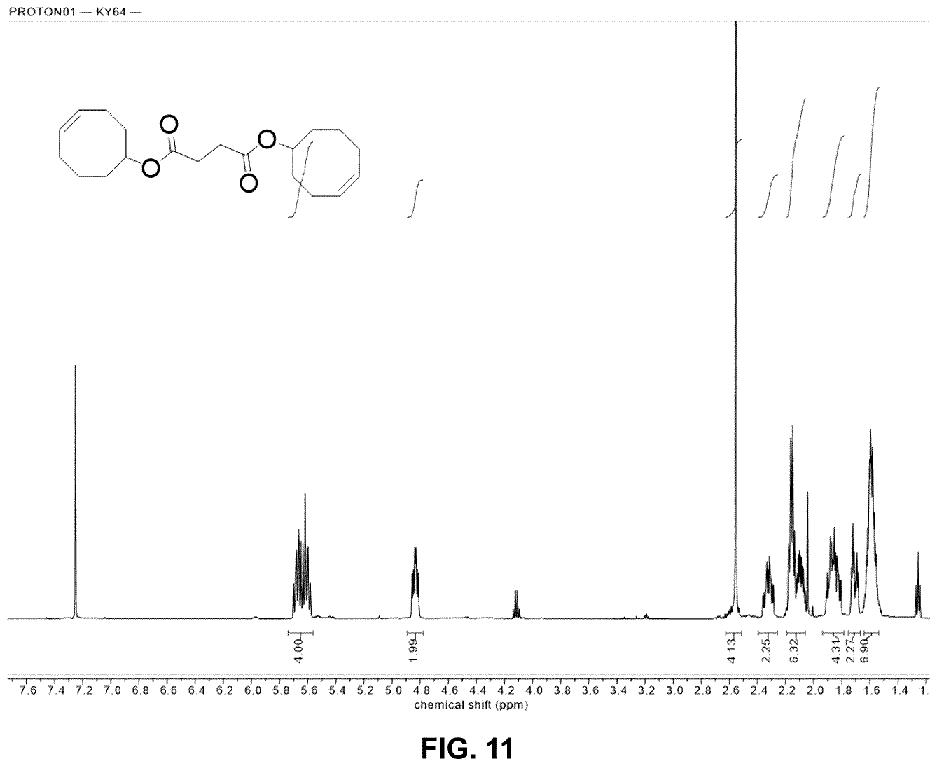

A proof-of-concept plug is constructed from a shape memory polymer composite, crosslinked poly(cis-cyclooctene) containing Fe₃O₄ nanoparticles (MK-1-7H), as follows. The material is cut into a rectangular strip of dimensions 3.45 mm×9.87 mm×1.37 mm. The material is then heated to Tₜᵣₐₙₛ by placing it in an oven at 130° C. for 5 minutes. While hot, the material is folded 180° and compressed using a poly(methyl methacrylate) screw clamp. Cooling to room temperature completed fabrication and shape memory programming of the prototype valve plug. The plug s tested in an apparatus (See FIG. 10) consisting of a focused ultrasound transducer (Precision Acoustics Model 960) operated at 30 V input voltage and 670 kHz and aimed upwards towards a sample stage in a temperature-controlled water tank, and a thermal camera (FLIR A655sc) was placed aiming downwards perpendicular to the sample. The sample stage holds a Petri dish with a Mylar film bottom that is transparent to ultrasound. The water level comes up to the bottom of the dish and the temperature was set to 37.0° C. H₂O is pipetted into the well in the bottom of the dish (˜1 mL) and the sample is placed in the thin layer of fluid. Upon ultrasound stimulation, the plug is observed to unfold within a period of ˜7 seconds (FIG. 9C). The temperature of the sample as recorded with the thermal camera rises to ˜50° C. quickly and is stable at that temperature during the period of shape change. After shape change is complete, the sample temperature continues to rise. Notably, the surrounding water did not change significantly in temperature throughout the process.

### Example 7: Synthesis of SMP Composites

To a 20 mL scintillation vial containing the additive (1-15 wt. % relative to cis-cyclooctene+crosslinker) and a stir bar in a N₂ filled glovebox is added 1.026 mL of a mixture containing cis-cyclooctene (1.000 mL, 7.673 mmol, 1.0 eq.) and crosslinker (0.026 mL, 0.0767 mmol, 0.01 eq.). While stirring, 6.000 mL of a stock solution containing 2,6-Di-tert-butyl-4-methylphenol (0.2 mg, 0.0008 mmol, 0.0001 eq.) and G2, Benzylidene[1,3-bis(2,4,6-trimethylphenyl)-2-imidazolidinylidene]dichloro(tricyclohexylphosphine)ruthenium, (0.65 mg, 0.00077 mmol, 0.0001 eq.) in dichloromethane (DCM). The solution is allowed to stir for 30 minutes, during which time gelation is observed. The vial is then removed from the glovebox and 6 mL of DCM containing 1 drop of ethyl vinyl ether is added under air. After 30 minutes, the solution above the gel is removed, and the gel is dried under vacuum on a Schlenk line for 48 hours to obtain a solid polymer sample, the color of which varied depending on the additive (FIG. 10).

Reagents used above were commercially acquired and used as received except for as described here. cis-cyclooctene was stirred over CaH₂ for 24 hours and then distilled under vacuum, followed by storage in a Schlenk flask under Ar atmosphere until use in the above procedure. The crosslinker is synthesized from 4-cyclooctenol and succinic acid as follows.

Succinic acid (1 eq., 0.468 g, 3.962 mmol) and 4-dimethylaminopyridine (0.1 eq., 0.050 g, 0.3962 mmol) are dissolved in DCM (15 mL) under air. Triethylamine (3 eq., 1.700 mL, 11.886 mmol) is added to the solution at room temperature. At 0° C., 1-Ethyl-3-(3′-dimethylaminopropyl)carbodiimide hydrochloride (EDC-HCl, 2.8 eq., 2.127 g, 11.094 mmol) is added, followed by addition of cis-cyclooct-4-en-1-ol (2 eq., 1.0 g, 7.924 mmol). The reaction mixture is stirred at room temperature overnight. The mixtures diluted with DCM (30 mL) and an aqueous extraction is performed with water and brine. The product is purified with column chromatography on silica gel. 60% yield was obtained as a colorless oil. Purity is supported with 1H nuclear magnetic resonance (NMR) spectroscopy. 1H NMR (500 MHz, CDCl₃) δ 5.74-5.56 (m, 4H), 4.83 (td, J=9.5, 4.3 Hz, 2H), 2.55 (s, 4H), 2.39-2.26 (m, 2H), 2.19-2.06 (m, 6H), 1.94-1.79 (m, 4H), 1.75-1.67 (m, 2H), 1.64-1.54 (m, 6H).

### Example 8: DSC Procedure

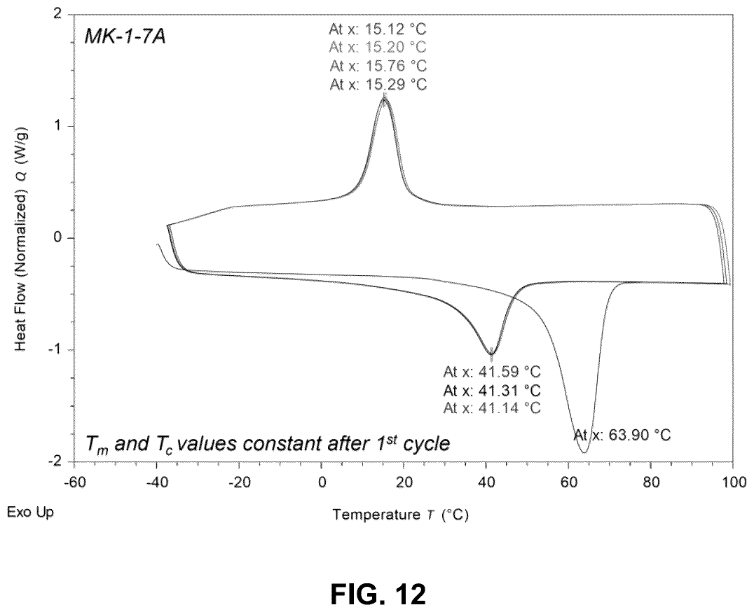

A dried polymer sample synthesized and weighing 7.6 mg is placed in an aluminum pan and hermetically sealed. DSC is performed at a ramp rate of 10° C./min for the indicated number of cycles between −40.0° C. and 100.0° C. (FIG. 12) using a TA instruments DSC-25. Air is used as the purge gas.

### Example 9: Relationship Between DSC Results and Sample Processing

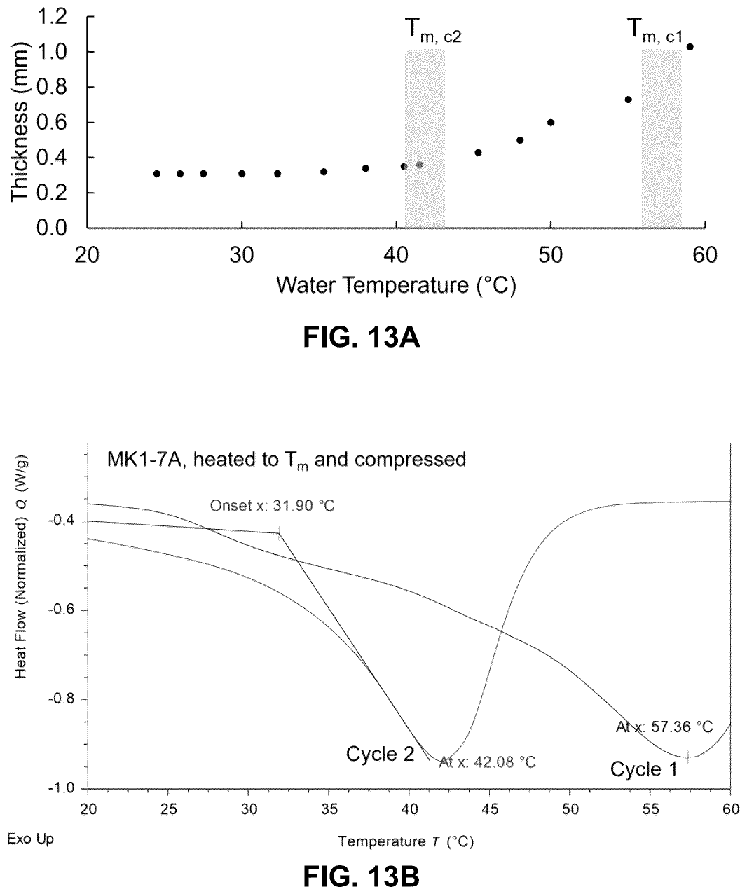

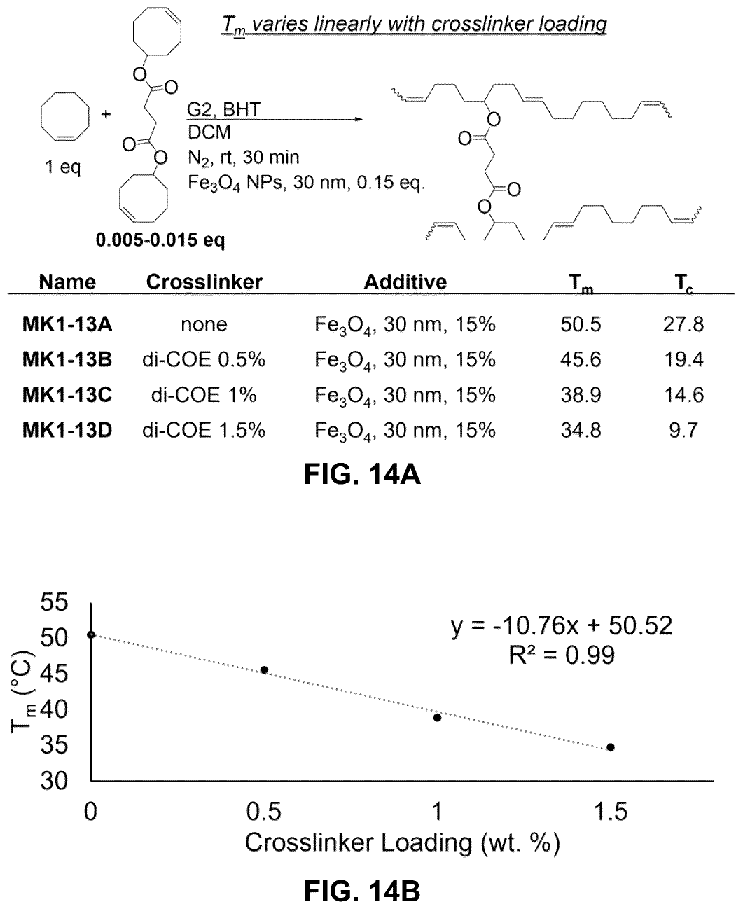

A sample prepared with no additive (MK-1-7A) is heated to Tₘ in a 60° C. metal bead bath and then compressed while hot in custom clamp (see above for details). The sample is allowed to cool to room temperature and was cut to a rectangular sheet for DSC (weight 18.7 mg). DSC is run to test the hypothesis that the Tₘ obtained from repeated DSC cycling is in fact the temperature at which shape memory occurs. The sample processed for DSC reflects the processing commonly used for shape memory testing. In a second experiment, a sample from the same batch of polymer is heated to Tₘ and compressed from thickness 1.83 mm to 0.33 mm using the custom clamp without the mold insert piece. The flattened sample is equilibrated for 5 minutes in a water bath at increasing temperatures and the thickness is measured afterwards using calipers. The resulting plot of thickness vs. temperature is compared with the DSC results (FIGS. 13A-13B). Notably, the shape memory effect does not begin until after the true Tₘ (cycle 2) is exceeded. A more significant change in thickness is observed as the temperature approaches the cycle 1 Tₘ, that obtained without erasure of the thermal history.

### Example 10: Tₘ is Controllable Through Varying Crosslinker Loading

Composite samples were synthesized as in [0046] with varying crosslinker loading from 0.5-1.5 mol % relative to monomer. Resulting samples were characterized by DSC and found to exhibit a linear relationship between Tₘ and crosslinker loading.

### Example 11: Ultrasound Induced Opening of Valve Prototype Based on Concept 2

Two prototype valves are constructed using a design based on Concept 2 featuring a 24/40 rubber septum which holds the valve plug sample using compression from a copper retaining wire. The septum has a hole punched in it (˜2 mm diameter) through which the balloon contents (fluid or air) can flow out after the valve plug is activated with ultrasound. The valve plug is prepared from a crosslinked poly(cis-cyclooctene) composite (MK⁻¹-21C) containing 1 wt. % K25 hollow glass microspheres (3M, K25 Glass Bubbles) as an ultrasound absorbing additive. The 2 plugs (cylindrical, d=˜5.0 mm, thickness=2.88 [plug 1], 2.71 [plug 2] mm) are programmed by heating to 70° C. in an oven and clamping the hot sample using the custom clamp apparatus described in Example 3. The samples are cooled to room temperature in the clamp and found to have the form of a disc (diameter=˜8.0 mm and thickness=1.68 [plug 1] and 1.58 [plug 2] mm). The programmed samples are each placed into the narrow end of a rubber septum with approx. 2 mm hole, and sealed in place with the retaining wire.

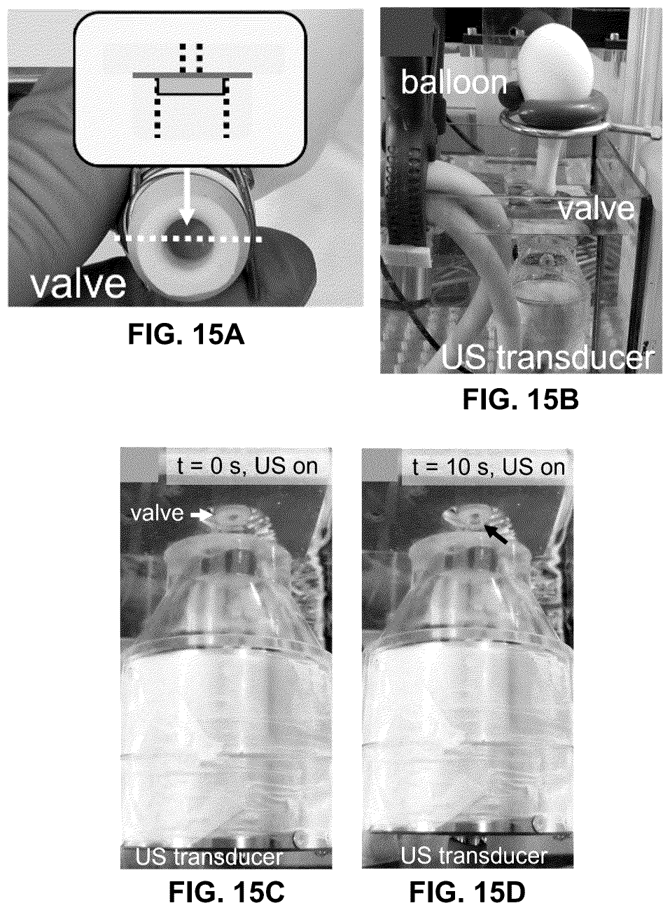

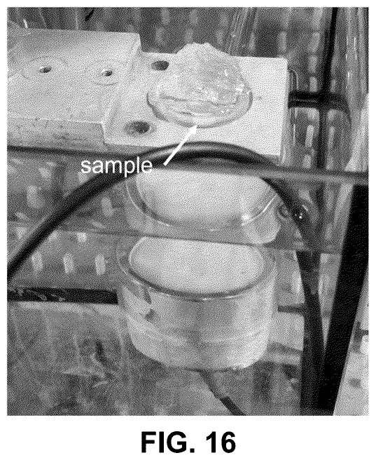

Balloons containing either water (with green food coloring for visualization, plug 1/device 1) or air (plug 2/device 2) are attached to the wide end of the septum and held in place with electrical tape. The completed balloon valve prototypes (6× scale compared to final application, insufflation valve omitted) are exposed to ultrasound and tested in the ultrasound setup described in Example 6, with the water bath set to 30° C. The dish is removed from the sample holder and a ring stand was added to position the prototypes in alignment with the transducer, with the valve ends submerged (see FIGS. 15A-15D). In the course of ultrasound exposure for 1 minute, the valves are observed to open in both cases, releasing water or air. The valve in device 2 opened at t=10 s ultrasound exposure as marked by the release of bubbles. The release rate is slow due to compression from the retaining wire, but this rate could be tuned by adjusting the tightness. The balloon for device 2 is observed to fully deflate. After testing, the valve plugs are removed and found to have undergone shape memory near to completion, with final thicknesses (2.72 [valve 1] and 2.69 [valve 2]) close to the original dimensions, giving high strain recovery ratios (0.87 [valve 1] and 0.96 [valve 2]), where strain recovery ratio Rᵣ is defined as:

$${R}_{r} ( 1 ) = \frac{{ε}_{m} - {ε}_{p}}{{ε}_{m}} where {ε}_{m} = \frac{{t}_{1} - {t}_{2}}{{t}_{1}} and {ε}_{p} = \frac{{t}_{1} - {t}_{3}}{{t}_{1}}$$

Here t₁ is the thickness of the permanent shape, t₂ is the thickness of the temporary shape, and t₃ is the thickness after the ultrasound experiment. An Rᵣ(1)=1 would indicate complete strain recovery or restoration of the permanent shape. Overall, this experiment represents successful ultrasound induced deflation of a balloon device at a depth of approx. 6 cm (distance from transducer to sample).

### Example 12: Ultrasound Induced Plug Shape Change in Coupling Gel

A valve plug is synthesized (MK⁻¹-21C) containing 1 wt. % of K25 hollow glass microspheres (HGMs) as the ultrasound absorbing additive. The plug (cylindrical, d=˜5.0 mm, thickness=2.71 mm) is programmed by heating to 70° C. in an oven and clamping the hot sample using the custom clamp apparatus described in Example 3. The sample is cooled to room temperature in the clamp and found to have the form of a disc (diameter=˜8.0 mm and thickness=1.71 mm). The programmed sample is then placed in the sample dish in the ultrasound setup described in Example 6. The water bath below the sample dish is set to 30.0° C. Instead of placing the sample in a thin layer of water, a thick (approx. 3 cm) layer of ultrasound coupling gel (Aquasonic® 100 Ultrasound Transmission Gel, Parker Laboratories) is placed in the dish and the sample was fully surrounded by the gel. After 1 minute of ultrasound exposure, the sample is removed and observed to have undergone shape change, with a final thickness of 2.33 and strain recovery ratio Rᵣ(1)=0.62 (a ratio of 1 indicates complete recovery). This experiment supports that shape change can occur in a tissue-like medium and that the surrounding air above the sample as in the usual setup is not required.

### Example 13: Temporary Shape Stability>12 Weeks

4 valve plug samples (MK⁻¹-41D1-4) are prepared as in Example 12 using the custom clamp apparatus and allowed to sit at room temperature for 12 weeks in their temporary shapes (average dimensions 1.73 mm thickness and 7.75 mm diameter). The final dimensions of the samples are not significantly different from the starting dimensions (specifically, they vary by an average amount of 0.01 mm (0.4%) in thickness and 0.02 mm (0.2%) in diameter), indicating that the temporary shape is stable for at least 12 weeks, longer than the required duration that the valve needs to remain closed while occluding the trachea.

### Example 14: Additional Synthetic Embodiments

Synthesis of MK1-21C: To a scintillation vial containing dicumyl peroxide (2.0 wt. % relative to poly(cis-cyclooctene, 1.000 g) was added poly(cis-cyclooctene) (Vestenamer, 1.000 g), an ultrasound absorbing additive (1 wt. % K25 HGMs, 3M), a thixotrope (Cabosil TS610, 1.0 wt. %), and 10 mL toluene under air at room temperature. The mixture was stirred for 48 hours at which point it was noted to be a heterogeneous viscous solution where only the additive remains undissolved. The vial was agitated until the suspension was homogeneously mixed and the solvent was immediately removed by rotary evaporation followed by drying for 48 hours on a Schlenk line. Dried polymer samples were placed in a suitable mold and crosslinked in a vacuum oven (VWR Symphony) set to 150° C. for approximately 43 hours. Crosslinked samples were characterized by differential scanning calorimetry (DSC). This sample has Tₘ=39.1° C. and T_{c}=11.4° C. as obtained from the second heating and cooling cycle, respectively.

Synthesis of MK⁻¹-41D1-4: To a scintillation vial containing poly(cis-cyclooctene) (Vestenamer, 1.000 g), an ultrasound absorbing additive (iM30K HGMs, 3M, 5.0 wt. %, relative to polymer), a thixotrope (Cabosil TS610, 1.0 wt. %), dibenzoyl peroxide (2.5 wt. %), and 10 mL toluene under air at room temperature. The mixture was stirred for 48 hours at which point it was noted to be a heterogeneous viscous solution where only the additive remains undissolved. The vial was agitated until the suspension was homogeneously mixed and the solvent was immediately removed by rotary evaporation followed by drying for 48 hours on a Schlenk line. Dried polymer samples were placed in a suitable mold and crosslinked in a vacuum oven (VWR Symphony) set to 140° C. for approximately 18 hours.

### REFERENCES CITED OR RELEVANT TO THE ABOVE

[1] Kirby E, Keijzer R. Congenital diaphragmatic hernia: current management strategies from antenatal diagnosis to long-term follow-up. Pediatr Surg Int. 2020 April; 36(4):415-429. doi: 10.1007/s00383-020-04625-z. Epub 2020 Feb. 18. PMID: 32072236.
[2] Yu L, Hernan R R, Wynn J, Chung W K. The influence of genetics in congenital diaphragmatic hernia. Semin Perinatol. 2020 February; 44(1):151169. doi: 10.1053/j.semperi.2019.07.008. Epub 2019 Aug. 1. PMID: 31443905.
[3] Kovler M L, Jelin E B. Fetal intervention for congenital diaphragmatic hernia. Semin Pediatr Surg. 2019 August; 28(4):150818. doi: 10.1053/j.sempedsurg.2019.07.001. Epub 2019 Jul. 18. PMID: 31451175.
[4] Deprest J A, Nicolaides K H, Benachi A, Gratacos E, Ryan G, Persico N, Sago H, Johnson A, Wielgoś M, Berg C, Van Calster B, Russo F M; TOTAL Trial for Severe Hypoplasia Investigators. Randomized Trial of Fetal Surgery for Severe Left Diaphragmatic Hernia. N Engl J Med. 2021 Jul. 8; 385(2):107-118. doi: 10.1056/NEJMoa2027030. Epub 2021 Jun. 8. PMID: 34106556.
[5] Delaey J, Dubruel P, Van Vlierberghe S. Shape-Memory Polymers for Biomedical Applications. Adv Func Mater. 2020; 30(44):1909047. doi: 10.1002/adfm.201909047.

### Example 15: Additional Aspects, Features, Components

Any of the aspects and embodiments disclosed herein is optionally combined with any component(s), feature(s), element(s), device(s), mechanism(s), structure(s), configuration(s), dimensions(s), composition(s), portion(s), method(s) or method step(s), or any combination thereof, found in the following references, each of which is incorporated herein by reference in its entirety:
Franano, et al., US Patent Pub. 2022/0031486A1;
Franano, et al., U.S. Pat. No. 10,537,451B2;
Franano, et al., US Patent Pub. 2021/0275187A1;
Franano, et al., U.S. Pat. No. 11,484,318B2;
Serbinenko, et al., U.S. Pat. No. 4,282,875A;
Tilson, et al., US Patent Pub. 2017/0050004A1;
Tilson, et al., U.S. Pat. No. 11,471,653B2;
Franano, et al., U.S. Pat. No. 11,033,275B2;
J. S. Baba, et al., “Characterization of a reversible thermally-actuated polymer-valve: A potential dynamic treatment for congenital diaphragmatic hernia”, 2018, PLoS ONE 13(12), e0209855, DOI:10.1371/journal.pone.0209855; and
N. Sananes, et al., “Evaluation of a new balloon for fetal endoscopic tracheal occlusion in the nonhuman primate model”, Prenatal Diagnosis, 2019, 39, 403-408, DOI:10.1002/pd.5445.

For example, some features, such as catheter details or catheter-balloon connectors, and steps useful and well-known in the art to facilitate manufacturing, implantation, delivery or positioning, and/or fluid injection into balloons may be found in one or more of the above listed and incorporated references and may be useful in combination with various aspects and embodiments disclosed herein.

### Certain Aspects and Embodiments

Various aspects are contemplated herein, several of which are set forth in the paragraphs below. It is explicitly contemplated that any aspect or portion thereof can be combined to form an aspect. In addition, it is explicitly contemplated that: any reference to aspect 1 includes reference to aspects 1a, 1 b, and/or 1c, and any combination thereof; any reference to Aspect 23 includes reference to Aspects 23a and 23b, and so on (any reference to an aspect includes reference to that aspect's lettered versions). Moreover, the terms “any preceding aspect” and “any one of the preceding aspects” means any aspect that appears prior to the aspect that contains such phrase (for example, the sentence “Aspect 32: The method or system of any preceding aspect . . . ” means that any aspect prior to aspect 32 is referenced, including letter versions, including aspects 1a through 31). For example, it is contemplated that, optionally, any material, method, or device of any the below aspects may be useful with or combined with any other aspect provided below. Further, for example, it is contemplated that any embodiment or aspect described above may, optionally, be combined with any of the below listed aspects.

Aspect 1a: An implantable, detachable, and removable medical device comprising:
a therapeutic balloon configured to perform a therapeutic activity inside a living subject; and
a focused-ultrasound activatable actuator operably connected to the therapeutic balloon;
wherein the actuator is capable of being activated between a closed state and an open state remotely via a focused ultrasound beam;
wherein the device is configured to be in an implantation state to facilitate implantation of the device in the living subject, in a therapeutic state to facilitate performance of the therapeutic activity, and subsequently in an expulsion state to facilitate expulsion of the medical device from the living subject; and
wherein the implantation state, therapeutic state, and expulsion state are different from each other.

Aspect 1 b: A method for treating a condition in a living subject, the method comprising:
implanting a medical device with the aid of an implant-accessory;
filling a therapeutic balloon with a biologically-benign fluid via the implant-accessory; and
detaching the implant-accessory from the medical device;
wherein the medical device is implantable, detachable, and removable and the medical device comprises:
a therapeutic balloon configured to perform a therapeutic activity inside a living subject; and
a focused-ultrasound activatable actuator operably connected to the therapeutic balloon;
wherein the actuator is capable of being activated between a closed state and an open state remotely via a focused ultrasound beam;
wherein the device is configured to be in an implantation state to facilitate implantation of the device in the living subject, in a therapeutic state to facilitate performance of the therapeutic activity, and subsequently in an expulsion state to facilitate expulsion of the medical device from the living subject; and
wherein the implantation state, therapeutic state, and expulsion state are different from each other.

Aspect 1c: A method for making the medical device of any one of the preceding claims, the method comprising:
operably attaching a polymer component having an ultrasound-absorbing shape memory polymer to the therapeutic balloon thereby forming the focused-ultrasound activatable actuator;
wherein the medical device is implantable, detachable, and removable and the medical device comprises:
a therapeutic balloon configured to perform a therapeutic activity inside a living subject; and
a focused-ultrasound activatable actuator operably connected to the therapeutic balloon;
wherein the actuator is capable of being activated between a closed state and an open state remotely via a focused ultrasound beam;
wherein the device is configured to be in an implantation state to facilitate implantation of the device in the living subject, in a therapeutic state to facilitate performance of the therapeutic activity, and subsequently in an expulsion state to facilitate expulsion of the medical device from the living subject; and
wherein the implantation state, therapeutic state, and expulsion state are different from each other.

Aspect 2: The medical device or method of any preceding Aspect, wherein the actuator is capable of being activated remotely via a focused ultrasound beam.

Aspect 3: The medical device or method of any preceding Aspect, wherein the actuator can be activated from a closed state to an open state, from the open state to the closed state, or both.

Aspect 4: The medical device or method of any preceding Aspect, wherein the therapeutic state comprises the actuator being in the closed state and the expulsion state comprises the actuator being in the open state.

Aspect 5: The medical device or method of any preceding Aspect, wherein the implantation state and therapeutic state are characterized by the actuator being in its closed state; and wherein activation of the actuator from its closed state to its open state activates the expulsion state of the device.

Aspect 6: The medical device or method of any preceding Aspect, wherein the device is in the implantation state when being implanted in the living subject, subsequently in the therapeutic state when performing the therapeutic activity in the living subject, and further subsequently in the expulsion state when being expelled from the living subject;
Aspect 7: The medical device or method of any preceding Aspect, wherein the therapeutic state is characterized by the therapeutic balloon having a larger volume and/or cross-sectional size than the same of the expulsion state of the device.

Aspect 8: The medical device or method of any preceding Aspect, wherein the therapeutic state is characterized by the therapeutic balloon being expanded or inflated and the expulsion state is characterized by the therapeutic balloon being contracted or deflated.

Aspect 9: The medical device or method of any preceding Aspect, wherein the implantation state is characterized by the medical device comprising an implant-accessory that is operably and detachably connected to the therapeutic component; wherein the implant-accessory is capable of providing a fluid into a lumen of the therapeutic balloon; wherein the implant accessory is detachable and removable without deflating the therapeutic balloon upon detachment and removal of the implant accessory.

Aspect 10: The medical device or method of any preceding Aspect, wherein the expulsion state is characterized by the medical device being free of and not operably connected to the implant-accessory.

Aspect 11: The medical device or method of any preceding Aspect, wherein the implant-accessory facilitates providing the medical device to a desired region of the living subject and the implant-accessory facilitates converting the device from the implantation state to the therapeutic state.

Aspect 12: The medical device or method of any preceding Aspect being configured to be disconnected from the implant-accessory after the therapeutic state of the device is activated/obtained.

Aspect 13: The medical device or method of any preceding Aspect, wherein the therapeutic state comprises the therapeutic balloon being inflated with a biologically-benign fluid; and wherein the expulsion state comprises the therapeutic balloon being free or mostly free of the biologically-benign fluid thereby the balloon being deflated.

Aspect 14: The medical device or method of any preceding Aspect, wherein the therapeutic state comprises the therapeutic balloon remaining inflated substantially only by a fluid pressure of the biologically-benign fluid inside the therapeutic balloon.

Aspect 15a: The medical device or method of any preceding Aspect, wherein the therapeutic state comprises the therapeutic balloon being fluidically sealed. Aspect 15b: The medical device or method of any preceding Aspect, wherein the therapeutic state comprises the therapeutic balloon being fluidically sealed and fluid within a lumen of the therapeutic balloon being fluidically isolated from surroundings external to the balloon.

Aspect 16: The medical device or method of any preceding Aspect, wherein the implantation state is characterized by the medical device comprising an implant-accessory that is operably and detachably connected to the therapeutic component; wherein the implant-accessory comprises a fluid-delivery tube capable of delivering a biologically-benign fluid directly or indirectly into a lumen of the therapeutic balloon; wherein the implant accessory is detachable and removable without deflating the therapeutic balloon upon detachment and removal of the implant accessory; and wherein the implant-accessory can be detached from the medical device after the device is implanted.

Aspect 17: The medical device or method of any preceding Aspect, wherein the actuator comprises a polymer component capable of absorbing focused ultrasound; wherein one or more characteristics of the polymer component change as a result of it absorbing the focused ultrasound; and wherein the actuator is activated as a result of the change of the one or more characteristics of the polymer component.

Aspect 18: The medical device or method of any preceding Aspect, wherein the polymer component comprises a shape change polymer capable of absorbing focused ultrasound; wherein the shape change polymer is at least partially heated by absorbing the focused ultrasound as a result of which the polymer component undergoes an ultrasound-induced shape change.

Aspect 19: The medical device or method of any preceding Aspect, wherein the polymer component is characterized by a component transition temperature (T_{cm,trans}); wherein the polymer component or one or more portions thereof undergo the shape change from a temporary shape to a permanent shape when the polymer component or said one or more portions thereof are heated to within 35° C. of T_{cm,trans} or a temperature approximately equal to or greater than T_{cm,trans}.

Aspect 20: The medical device or method of any preceding Aspect, wherein T_{cm,trans} is selected from the range of approximately 40° C. (optionally 35° C., optionally 36° C., optionally 37° C., optionally 38° C., optionally 39° C., optionally 40° C.) to approximately 100° C. (optionally 45° C., optionally 50° C., optionally 55° C., optionally 60° C., optionally 65° C., optionally 70° C., optionally 75° C., optionally 80° C., optionally 85° C., optionally 90° C., optionally 95° C., optionally 100° C.), wherein any value and range therebetween inclusively is explicitly contemplated herein, such as approximately 45° C. to approximately 55° C.

Aspect 21: The medical device or method of any preceding Aspect, wherein the ultrasound-induced shape change is an expansion, a contraction, a twisting, an unraveling, a curling, an unfurling, an opening, a closing, a bending, an unbending, a folding, an unfolding, a straightening, a lengthening, a shortening, a redistribution or change in distribution of stress in the material, a redistribution or change in distribution of strain in the material, or any combination of these.

Aspect 22: The medical device or method of any preceding Aspect, wherein the ultrasound-induced shape change occurs as a result of exposure of the composite material or the one or more portions thereof to ultrasound characterized by frequencies selected from the range of approximately 300 kHz to approximately 3 MHz and an energy intensity selected from the range of approximately 1 W/cm² to approximately 3 W/cm².

Aspect 23a: The medical device or method of any preceding Aspect, wherein the polymer component is an ultrasound-absorbing material characterized an ultrasound attenuation coefficient of at least 0.16 dB/mm (optionally at least 0.17 dB/mm, optionally at least 0.18 dB/mm, optionally at least 0.19 dB/mm, optionally at least 0.20 dB/mm, optionally at least 0.21 dB/mm, optionally at least 0.22 dB/mm, optionally at least 0.23 dB/mm, optionally at least 0.24 dB/mm, optionally at least 0.25 dB/mm, optionally at least 0.26 dB/mm, optionally at least 0.27 dB/mm, optionally at least 0.28 dB/mm, optionally at least 0.29 dB/mm, optionally at least 0.30 dB/mm, optionally at least 0.31 dB/mm, optionally at least 0.32 dB/mm, optionally at least 0.33 dB/mm, optionally at least 0.34 dB/mm, optionally at least 0.35 dB/mm, optionally at least 0.37 dB/mm, optionally at least 0.40 dB/mm, optionally at least 0.42 dB/mm, optionally at least 0.45 dB/mm, optionally at least 0.47 dB/mm, optionally at least 0.50 dB/mm, optionally at least 0.51 dB/mm, optionally at least 0.52 dB/mm, optionally at least 0.53 dB/mm, optionally at least 0.54 dB/mm, optionally at least 0.55 dB/mm, optionally at least 0.56 dB/mm, optionally at least 0.57 dB/mm, optionally at least 0.58 dB/mm, optionally at least 0.59 dB/mm, optionally at least 0.60 dB/mm, optionally at least 0.9 dB/mm, optionally at least 1.0 dB/mm, optionally at least 1.2 dB/mm, optionally at least 1.5 dB/mm, optionally at least 1.75 dB/mm, optionally at least 2.0 dB/mm, optionally at least 2.25 dB/mm, optionally at least 2.50 dB/mm, optionally at least 2.75 dB/mm, optionally at least 3.0 dB/mm, optionally at least 3.25 dB/mm, optionally at least 3.5 dB/mm, optionally at least 3.75 dB/mm) at ultrasound frequencies selected from the range of 400 kHz to 600 kHz (optionally at 500 kHz). Aspect 23b: The medical device or method of any preceding Aspect, wherein the polymer component is an ultrasound-absorbing material characterized an ultrasound attenuation coefficient selected from the range of 0.15 dB/mm (optionally 0.5 dB/mm) to 10 dB/mm (optionally 5 dB/mm), wherein any value and range therebetween inclusively is explicitly contemplated.

Aspect 24: The medical device or method of any preceding Aspect, wherein shape memory polymer comprises polycyclooctene (PCOE), polycaprolactone (PCL), poly(lactic acid)(PLA), poly(lactic-co-glycolic acid)(PLGA), polyethylene (PE), polypropylene (PP), and thermoplastic polyurethane (TPU), or any combination thereof.

Aspect 25: The medical device or method of any preceding Aspect, wherein the shape memory polymer comprises a crosslinking moiety derived from a crosslinking precursor selected from the group consisting of: a di(4-cyclooctenol) succinate, dicumyl peroxide (DCP), dibenzoyl peroxide (DBzP), di(tert-butyl) peroxide, any derivative thereof, any analogue thereof, and any combination thereof.

Aspect 26: The medical device or method of any preceding Aspect, wherein the actuator is a valve for retaining or releasing a biologically-benign fluid from the therapeutic balloon.

Aspect 27: The medical device or method of Aspect 26, wherein the open state of the actuator is configured to allow the biologically-benign fluid to pass therethrough and the closed state of the actuator is configured to block passage of the biologically-benign fluid therethrough.

Aspect 28: The medical device or method of Aspect 26 or 27, wherein the actuator comprises a fluid-escape conduit and the polymer component is a plug; and
wherein the closed state of the actuator is characterized by the plug blocking fluid flow through the fluid-escape conduit, thereby retaining the biologically-benign fluid in the therapeutic balloon.

Aspect 29: The medical device or method of any of Aspects 26-28, wherein the therapeutic state comprises the actuator being in the closed state; and wherein the ultrasound-induced shape change of the polymer component activates the open state of the actuator.

Aspect 30: The medical device or method of any of Aspects 28-29, wherein the ultrasound-induced shape change of the polymer component activates the open state of the actuator thereby allowing the biologically-benign fluid to flow out of the therapeutic balloon and through the fluid conduit further thereby deflating the therapeutic balloon and activating the expulsion state of the device.

Aspect 31: The medical device or method of any of Aspects 26-30, wherein the biologically-benign fluid is released into the living subject when the actuator is open.

Aspect 32: The medical device or method of any of Aspects 28-31, wherein the plug is characterized by a component transition temperature (T_{cm,trans}); wherein the plug undergoes an ultrasound-induced shape change from a temporary shape to a permanent shape when the plug or one or more portions thereof are heated to within 35° C. of T_{cm,trans} or a temperature approximately equal to or greater than T_{cm,trans} as a result of the plug or said one or more portions thereof absorbing the focused ultrasound; and
wherein the actuator is closed if the plug has the temporary shape and the actuator is open if the plug has the permanent shape.

Aspect 33: The medical device or method of any of Aspects 28-32, wherein the ultrasound-induced shape change is an expansion of the plug such that it is at least partially released from the fluid-escape conduit allowing the biologically-benign fluid to flow through the fluid-escape conduit.

Aspect 34: The medical device or method of any of Aspects 28-33, wherein the temporary shape comprises a cylinder having a diameter approximately equal to a diameter of the fluid-escape conduit; and wherein the permanent shape has a cross-sectional dimension greater than the diameter of the fluid-escape conduit.

Aspect 35: The medical device or method of any of Aspects, wherein the ultrasound-induced shape change is a decrease in cross-sectional dimension of the plug from greater than a diameter of the fluid-escape conduit to less than the diameter of the fluid-escape conduit.

Aspect 36 The medical device or method of any of Aspects 28-32 or 35, wherein the temporary shape comprises a disk or cylinder having a diameter greater than a diameter of the fluid-escape conduit; and wherein the permanent shape comprises a disk or cylinder having a diameter less than the diameter of the fluid-escape conduit.

Aspect 37: The medical device or method of any of Aspects 28-32, wherein the ultrasound-induced shape change comprises formation of a hole or opening in the plug through which the biologically-benign fluid mass pass toward the fluid-escape element.

Aspect 38: The medical device or method of any of Aspects 28-32 or 37, wherein the temporary shape comprises a solid disk or cylinder free of a hole or opening in its cross-sectional shape; and wherein the permanent shape comprises a hole or opening therethrough or wherein the permanent shape is characterized by a cross-sectional shape of an annulus.

Aspect 39: The medical device or method of any of Aspects 28-32, wherein the ultrasound-induced shape change comprises an unfolding which forms a fluid pathway.

Aspect 40: The medical device or method of any of Aspects 28-32 or 39, wherein the temporary shape comprises a folded sheet with a hole or opening that is fluidically sealed by another portion of the temporary shape; wherein the permanent shape comprises an unfolded sheet having the hole or opening being fluidically open; and wherein the shape change is an unfolding.

Aspect 41: The medical device or method of any of Aspects 27-40, wherein the actuator and the polymer component thereof are configured to remain physically connected to or a part of the medical device in the implantation state, in the therapeutic state, and in the expulsion state.

Aspect 42: The medical device or method of any preceding Aspect, wherein the therapeutic state and expulsion state are characterized by the device being free of and physically disconnected from an implant-accessory or any component that is at least partially external to the living subject.

Aspect 43: The medical device or method of any preceding Aspect being configured to remain implanted in the living subject in the therapeutic state for at least 12 hours.

Aspect 44: The medical device or method of any preceding Aspect, wherein the therapeutic state is characterized by the therapeutic balloon having an inflated diameter selected from the range of 6 mm to 12 mm, optionally, 1 mm to 30 mm, wherein any value and range therebetween inclusively is explicitly contemplated herein, such as optionally 6.5 mm to 8.5 mm, or any other medically-useful value or range.

Aspect 45: The medical device or method of any preceding Aspect, wherein the therapeutic state is characterized by the therapeutic balloon having a length selected from the range of 10 mm to 30 mm, wherein any value and range therebetween inclusively is explicitly contemplated herein, such as optionally 20 mm to 24 mm, or any other medically-useful value or range.

Aspect 46: The medical device or method of any preceding Aspect, wherein the expulsion state is characterized by the therapeutic balloon having a deflated diameter selected from the range of 0.5 mm to 2 mm, optionally 0.1 mm to 5 mm, wherein any value and range therebetween inclusively is explicitly contemplated herein, such as optionally mm to 1.5 mm, or any other medically-useful value or range.

Aspect 47: The medical device or method of any preceding Aspect, wherein the therapeutic state is characterized by the therapeutic balloon being inflated and having a cylindrical shape and/or circular cross-section during the entirety of the existence of the therapeutic state.

Aspect 48a: The medical device or method of any preceding Aspect, wherein the therapeutic balloon has a wall thickness selected from the range of range of 0.005 mm to 0.5 mm, and wherein any value and range therebetween is explicitly contemplated herein. Aspect 48b: The medical device or method of any preceding Aspect, wherein the therapeutic balloon has a wall thickness selected from the range of range of 0.015 mm to 0.06 mm, and wherein any value and range therebetween is explicitly contemplated herein, or any other medically-useful value or range.

Aspect 49: The medical device or method of any preceding Aspect, wherein the device is configured to be self-expelled via a natural bodily process of the living subject and does not require an invasive removal procedure.

Aspect 50: The medical device or method of any preceding Aspect, wherein the device is an occlusion device and the therapeutic activity is an occlusion.

Aspect 51: The medical device or method of any preceding Aspect, wherein the occlusion device is configured to occlude an anatomical vessel, tube, or capillary in a living subject.

Aspect 52: The medical device or method of any preceding Aspect, wherein the therapeutic activity is tracheal occlusion.

Aspect 53: The medical device or method of any preceding Aspect, wherein the medical device is a deflatable endoscopic detachable balloon.

Aspect 54: The medical device or method of any preceding Aspect, wherein the living subject is an infant or a fetus.

Aspect 55: The medical device or method of any preceding Aspect, wherein the therapeutic state comprises the therapeutic balloon being inflated with a biologically-benign fluid; and wherein the therapeutic biologically-benign fluid comprises a saline solution or water.

Aspect 56: The medical device or method of any preceding Aspect, wherein the therapeutic state comprises the therapeutic balloon being inflated with a biologically-benign fluid; and wherein the biologically-benign fluid is radiopaque and/or is detectable via magnetic resonance imaging.

Aspect 57: The medical device or method of any preceding Aspect, wherein the therapeutic state comprises the therapeutic balloon being inflated with a biologically-benign fluid; and wherein the biologically-benign fluid comprises a therapeutic drug.

Aspect 58: The medical device or method of any preceding Aspect, wherein the balloon is formed of a biologically-compatible material.

Aspect 59: The medical device or method of any preceding Aspect, wherein the polymer component is formed of and/or is coated by a biologically-compatible material.

Aspect 60: The medical device or method of any preceding Aspect comprising a locking mechanism capable of connecting and disconnecting an implant-accessory from the therapeutic balloon; wherein the locking mechanism provides a fluidic connection between the therapeutic balloon and the implant-accessory when connected in the implantation state.

Aspect 61: The medical device or method of any preceding Aspect being free of electronics, a sensor, a battery, electrical wires, or any combination thereof.

Aspect 62: A method for treating a condition in a living subject, the method comprising:
implanting the medical device of any of the preceding Aspects with the aid of an implant-accessory;
filling the therapeutic balloon with a biologically-benign fluid via the implant-accessory; and
detaching the implant-accessory from the medical device.

Aspect 63: The method of Aspect 62 comprising leaving the medical device in the living subject for at least 12 hours.

Aspect 64: The method of Aspect 62 or 63 not comprising an invasive removal procedure for removing the medical device from the living subject.

Aspect 65: The method of any of Aspects 62-64, wherein the therapeutic activity is occlusion.

Aspect 66: The method of any of Aspects 62-65 being fetoscopic endoluminal tracheal occlusion.

Aspect 67: A method for making the medical device of any of the preceding Aspects, the method comprising:
operably attaching a polymer component having an ultrasound-absorbing shape memory polymer to the therapeutic balloon thereby forming the focused-ultrasound activatable actuator.

Aspect 68: The method of Aspect 67, comprising operably connecting the therapeutic balloon to an implant-accessory.

Aspect 69: The method of any of Aspects 67 or 68, comprising setting a temporary shape of the polymer component to facilitate the actuator being in a closed state prior to operably attaching the polymer component.

Aspect 70: The medical device or method of any of the preceding Aspects, comprising one or more component(s), one or more feature(s), one or more element(s), one or more device(s), one or more mechanism(s), one or more structure(s), one or more configuration(s), one or more dimensions(s), one or more composition(s), one or more portion(s), one or more method(s) or method step(s), or any combination thereof, found in the following references, each of which is incorporated herein by reference in its entirety:
Franano, et al., US Patent Pub. 2022/0031486A1;
Franano, et al., U.S. Pat. No. 10,537,451B2;
Franano, et al., US Patent Pub. 2021/0275187A1;
Franano, et al., U.S. Pat. No. 11,484,318B2;
Serbinenko, et al., U.S. Pat. No. 4,282,875A;
Tilson, et al., US Patent Pub. 2017/0050004A1;
Tilson, et al., U.S. Pat. No. 11,471,653B2;
Franano, et al., U.S. Pat. No. 11,033,275B2;
J. S. Baba, et al., “Characterization of a reversible thermally-actuated polymer-valve: A potential dynamic treatment for congenital diaphragmatic hernia”, 2018, PLoS ONE 13(12), e0209855, DOI:10.1371/journal.pone.0209855; and
N. Sananes, et al., “Evaluation of a new balloon for fetal endoscopic tracheal occlusion in the nonhuman primate model”, Prenatal Diagnosis, 2019, 39, 403-408, DOI:10.1002/pd.5445.

Aspect 71: The medical device or method of any of the preceding Aspects, wherein the actuator, or the polymer component thereof, comprises a composite material having:
one or more shape memory polymers; and
a first additive provided in the shape memory polymer(s); wherein:
the first additive is a plurality of inorganic particles;
the composite material is characterized by a composite transition temperature (T_{cm,trans}); and
the composite material or one or more portions thereof undergo a shape change from a temporary shape to a permanent shape when the composite material or said one or more portions thereof are heated to within 35° C. of T_{cm,trans} or a temperature approximately equal to or greater than T_{cm,trans}.

Aspect 72: The medical device or method of any of the preceding Aspects, wherein the actuator, or the polymer component thereof, comprises a composite material having:
one or more shape memory polymers; and
a first additive provided in the shape memory polymer(s); wherein:
the first additive increases one or more ultrasound-attenuation characteristics of the composite material same one or more shape memory polymers free of said first additive;
the composite material is characterized by a composite transition temperature (T_{cm,trans}); and
the composite material or one or more portions thereof undergo a shape change from a temporary shape to a permanent shape when the composite material or said one or more portions thereof are heated to within 35° C. (optionally within 30° C., optionally within 25° C., optionally within 20° C., optionally within 15° C., optionally within 10° C., optionally within 5° C.) of T_{cm,trans} or a temperature approximately equal to (e.g., “approximately equal”=within 20%, optionally within 10%, optionally within 5%, optionally within 1%, optionally equal) or greater than T_{cm,trans}.

Aspect 73: The medical device or method of any of the preceding Aspects, wherein the actuator, or the polymer component thereof, comprises a composite material having:
one or more shape memory polymers;
a first additive provided in the shape memory polymer(s); wherein:
the first additive is a plurality of hollow particles;
the composite material is characterized by a composite transition temperature (T_{cm,trans}); and

the composite material or one or more portions thereof undergo a shape change from a temporary shape to a permanent shape when the composite material or said one or more portions thereof are heated to within 35° C. of T_{cm,trans} or a temperature approximately equal to or greater than T_{cm,trans}.

Aspect 74: A method of using a composite material, the method comprising:
directing one or more focused ultrasound beams at one or more portions of the composite material;
thereby, heating the one or more portions to a temperature approximately equal to or greater than a composite transition temperature (T_{cm,trans}); and
thereby, causing the composite material to undergo a shape change at the one or more portions thereof;
wherein the composite material comprises:
   one or more shape memory polymers; and
   a first additive provided in the shape memory polymer(s); wherein:
   the first additive (a) is a plurality of inorganic particles, (b) increases an ultrasound-attenuation characteristic of the composite material compared to same one or more shape memory polymers free of said first additive, and/or (c) is a plurality of hollow particles;
   the composite material is characterized by the composite transition temperature (T_{cm,trans}); and
   the one or more portions of the composite material undergo the shape change from a temporary shape to a permanent shape when the one or more portions are heated to within 35° C. (optionally within 30° C., optionally within 25° C., optionally within 20° C., optionally within 15° C., optionally within 10° C., optionally within 5° C.) of T_{cm,trans} or a temperature approximately equal to (e.g., “approximately equal”=within 20%, optionally within 10%, optionally within 5%, optionally within 1%, optionally equal) or greater than T_{cm,trans}.

Aspect 75: A method of making a composite material, the method comprising:
polymerizing a monomer to form a first polymer;
crosslinking the first polymer in the presence of a crosslinking precursor and the first additive at a temperature approximately (e.g., within 20%) equal to or greater than a crosslinking temperature (T_{cm,crosslink}) (optionally at a temperature greater than or equal to 65° C., optionally a temperature greater than or equal to 75° C., a temperature greater than or equal to 85° C., a temperature greater than or equal to 90° C., a temperature greater than or equal to 95° C., a temperature greater than or equal to 100° C., a temperature greater than or equal to 105° C., a temperature greater than or equal to 110° C., a temperature greater than or equal to 115° C.) to form the composite material having the crosslinked shape memory polymer and the first additive;
wherein the composite material comprises:
   one or more shape memory polymers; and
   a first additive provided in the shape memory polymer(s); wherein:
   the first additive (a) is a plurality of inorganic particles, (b) increases an ultrasound-attenuation characteristic of the composite material compared to same one or more shape memory polymers free of said first additive, and/or (c) is a plurality of hollow particles;
   the composite material is characterized by the composite transition temperature (T_{cm,trans}); and
   the one or more portions of the composite material undergo the shape change from a temporary shape to a permanent shape when the one or more portions are heated to within 35° C. (optionally within 30° C., optionally within 25° C., optionally within 20° C., optionally within 15° C., optionally within 10° C., optionally within 5° C.) of T_{cm,trans} or a temperature approximately equal to (e.g., “approximately equal”=within 20%, optionally within 10%, optionally within 5%, optionally within 1%, optionally equal) or greater than T_{cm,trans}.

Aspect 76: A method of making a device having a composite material, the method comprising:
attaching, providing, or inserting the composite material to or into the device;
wherein the composite material comprises:
   one or more shape memory polymers; and
   a first additive provided in the shape memory polymer(s); wherein:
   the first additive (a) is a plurality of inorganic particles, (b) increases an ultrasound-attenuation characteristic of the composite material compared to same one or more shape memory polymers free of said first additive, and/or (c) is a plurality of hollow particles;
   the composite material is characterized by the composite transition temperature (T_{cm,trans}); and
   the one or more portions of the composite material undergo the shape change from a temporary shape to a permanent shape when the one or more portions are heated to within 35° C. (optionally within 30° C., optionally within 25° C., optionally within 20° C., optionally within 15° C., optionally within 10° C., optionally within 5° C.) of T_{cm,trans} or a temperature approximately equal to (e.g., “approximately equal”=within 20%, optionally within 10%, optionally within 5%, optionally within 1%, optionally equal) or greater than T_{cm,trans}.

Aspect 77: A device comprising: a composite material, wherein the composite material comprises:
one or more shape memory polymers; and
a first additive provided in the shape memory polymer(s); wherein:
the first additive (a) is a plurality of inorganic particles, (b) increases an ultrasound-attenuation characteristic of the composite material compared to that of the shape memory polymer(s) alone, and/or (c) is a plurality of hollow particles;
the composite material is characterized by the composite transition temperature (T_{cm,trans}); and
the one or more portions of the composite material undergo the shape change from a temporary shape to a permanent shape when the one or more portions are heated to within 35° C. (optionally within 30° C., optionally within 25° C., optionally within 20° C., optionally within 15° C., optionally within 10° C., optionally within 5° C.) of T_{cm,trans} or a temperature approximately equal to (e.g., “approximately equal”=within 20%, optionally within 10%, optionally within 5%, optionally within 1%, optionally equal) or greater than T_{cm,trans}.

Aspect 78: The medical device or method of any of the preceding Aspects, wherein the actuator, or the polymer component thereof, comprises a composite material having:
one or more shape memory polymers; and
a first additive provided in the one or more shape memory polymers; wherein:
(a) the first additive is a plurality of inorganic particles, (b) the first additive increases an ultrasound attenuation coefficient of the composite material compared to that of the same one or more shape memory polymers free of said first additive; and/or (c) the first additive is a plurality of hollow particles; the composite material is characterized by a composite transition temperature (T_{cm,trans});
the first additive is provided at least at one or more portions of the composite material; and
the composite material or the one or more portions thereof undergo a shape change from a temporary shape to a permanent shape when the composite material or said one or more portions thereof are heated to within 35° C. (optionally within 30° C., optionally within 25° C., optionally within 20° C., optionally within 15° C., optionally within 10° C., optionally within 5° C.) of T_{cm,trans} or a temperature approximately equal to (e.g., “approximately equal”=within 20%, optionally within 10%, optionally within 5%, optionally within 1%, optionally equal) or greater than T_{cm,trans}.

Aspect 79a: The composite material, method, and/or device of any preceding Aspect, wherein the first additive is provided at (or at least at) the one or more portions of the shape memory polymer(s). Aspect 79b: The composite material, method, and/or device of any preceding Aspect, wherein the first additive is provided at the one or more portions of the shape memory polymer(s); and wherein at least a portion of the one or more shape memory polymers is free of the first additive. Aspect 79c: The composite material, method, and/or device of any preceding Aspect, wherein the first additive is provided at (or at least at) one or more portions of the composite material. Aspect 79d: The composite material, method, and/or device of any preceding Aspect, wherein the first additive is provided at one or more portions of the composite material; and wherein at least another portion of the one or more shape memory polymers is free of the first additive.

Aspect 80: The composite material, method, and/or device of any preceding Aspect, wherein the first additive is provided throughout the internal volume of the shape memory polymer(s).

Aspect 81: The composite material, method, and/or device of any preceding Aspect being capable of absorbing ultrasound throughout or at the one or more portions of the composite material; wherein a temperature of the composite material increases where its absorbs the ultrasound; and wherein the composite material undergoes the shape change when heated to within 35° C. of T_{cm,trans} or a temperature approximately equal to or greater than T_{cm,trans} as a result of absorbing of the ultrasound.

Aspect 82: The composite material, method, and/or device of any preceding Aspect, wherein the one or more portions of the composite material having the first additive are heated when said one or more portions of the composite material are exposed to ultrasound.

Aspect 83: The composite material, method, and/or device of any preceding Aspect, wherein the first additive absorbs the ultrasound frequencies; and wherein the first additive is heated by its absorption of ultrasound and/or wherein heat is created by friction between the first additive and the one or more shape memory polymers when the first additive absorbs the ultrasound.

Aspect 84: The composite material, method, and/or device of any preceding Aspect, wherein the composite material undergoes the shape change only at the one or more portions thereof having the first additive exposed to the ultrasound.

Aspect 85a: The composite material, method, and/or device of any preceding Aspect, wherein the first additive increases one or more ultrasound-attenuation characteristic (optionally, an ultrasound absorption coefficient) of the composite material and/or of the one or more portions thereof (having the first additive) by at least 50% (optionally at least 75%, optionally at least 90%, optionally at least 100%, optionally at least 120%, optionally at least 140%, optionally at least 160%, optionally at least 180%, optionally at least 200%, optionally at least 220%, optionally at least 240%, optionally at least 260%, optionally at least 280%, optionally at least 300%, optionally at least 320%, optionally at least 340%) compared to that of the same one or more shape memory polymers free of said first additive (additive-free crosslinked shape memory polymer).

Aspect 85b: The composite material, method, and/or device of any preceding Aspect, wherein the first additive increases an ultrasound absorption coefficient of the composite material and/or of the one or more portions thereof (having the first additive) by at least 50% (optionally at least 75%, optionally at least 90%, optionally at least 100%, optionally at least 120%, optionally at least 140%, optionally at least 160%, optionally at least 180%, optionally at least 200%, optionally at least 220%, optionally at least 240%, optionally at least 260%, optionally at least 280%, optionally at least 300%, optionally at least 320%, optionally at least 340%) compared to that of the same one or more shape memory polymers free of said first additive (additive-free crosslinked shape memory polymer). Aspect 85c: The composite material, method, and/or device of any preceding Aspect, wherein the first additive increases one or more ultrasound-attenuation characteristic (optionally, an ultrasound absorption coefficient) of the composite material and/or the one or more portions thereof (having the first additive) by at least 50% (optionally at least 75%, optionally at least 90%, optionally at least 100%, optionally at least 120%, optionally at least 140%, optionally at least 160%, optionally at least 180%, optionally at least 200%, optionally at least 220%, optionally at least 240%, optionally at least 260%, optionally at least 280%, optionally at least 300%, optionally at least 320%, optionally at least 340%) compared to that of same one or more shape memory polymers free of said first additive (additive-free crosslinked shape memory polymer) at ultrasound frequencies selected from the range of 400 kHz to 600 kHz (optionally at 500 kHz). Aspect 85d: The composite material, method, and/or device of any preceding Aspect, wherein the first additive increases an ultrasound absorption coefficient of the composite material and/or the one or more portions thereof (having the first additive) by at least 50% (optionally at least 75%, optionally at least 90%, optionally at least 100%, optionally at least 120%, optionally at least 140%, optionally at least 160%, optionally at least 180%, optionally at least 200%, optionally at least 220%, optionally at least 240%, optionally at least 260%, optionally at least 280%, optionally at least 300%, optionally at least 320%, optionally at least 340%) compared to that of the same one or more shape memory polymers free of said first additive (additive-free crosslinked shape memory polymer) at ultrasound frequencies selected from the range of 400 kHz to 600 kHz (optionally at 500 kHz).

Aspect 86a: The composite material, method, and/or device of any preceding Aspect, wherein the one or more ultrasound-absorption characteristic of the composite material or of the one or more portions of the composite material having the first additive is an ultrasound attenuation coefficient and/or an ultrasound absorption characteristic. Aspect 86b: The composite material, method, and/or device of any preceding Aspect, wherein the one or more ultrasound-absorption characteristics of the composite material or of the one or more portions of the composite material having the first additive is an ultrasound attenuation coefficient.

Aspect 89a: The composite material, method, and/or device of any preceding Aspect, wherein the composite material and/or at least the one or more portions thereof (having the first additive) are characterized by an ultrasound attenuation coefficient selected from the range of 0.05 dB/mm to 10 dB/mm. Aspect 89b: The composite material, method, and/or device of any preceding Aspect, wherein the composite material and/or the one or more portions thereof (having the first additive) is characterized by an ultrasound attenuation coefficient of at least 0.16 dB/mm (optionally at least 0.17 dB/mm, optionally at least 0.18 dB/mm, optionally at least 0.19 dB/mm, optionally at least 0.20 dB/mm, optionally at least 0.21 dB/mm, optionally at least 0.22 dB/mm, optionally at least 0.23 dB/mm, optionally at least 0.24 dB/mm, optionally at least 0.25 dB/mm, optionally at least 0.26 dB/mm, optionally at least 0.27 dB/mm, optionally at least 0.28 dB/mm, optionally at least 0.29 dB/mm, optionally at least 0.30 dB/mm, optionally at least 0.31 dB/mm, optionally at least 0.32 dB/mm, optionally at least 0.33 dB/mm, optionally at least 0.34 dB/mm, optionally at least 0.35 dB/mm, optionally at least 0.37 dB/mm, optionally at least 0.40 dB/mm, optionally at least 0.42 dB/mm, optionally at least 0.45 dB/mm, optionally at least 0.47 dB/mm, optionally at least 0.50 dB/mm, optionally at least 0.51 dB/mm, optionally at least 0.52 dB/mm, optionally at least 0.53 dB/mm, optionally at least 0.54 dB/mm, optionally at least 0.55 dB/mm, optionally at least 0.56 dB/mm, optionally at least 0.57 dB/mm, optionally at least 0.58 dB/mm, optionally at least 0.59 dB/mm, optionally at least 0.60 dB/mm, optionally at least 0.9 dB/mm, optionally at least 1.0 dB/mm, optionally at least 1.2 dB/mm, optionally at least 1.5 dB/mm, optionally at least 1.75 dB/mm, optionally at least 2.0 dB/mm, optionally at least 2.25 dB/mm, optionally at least 2.50 dB/mm, optionally at least 2.75 dB/mm, optionally at least 3.0 dB/mm, optionally at least 3.25 dB/mm, optionally at least 3.5 dB/mm, optionally at least 3.75 dB/mm) at ultrasound frequencies selected from the range of 400 kHz to 600 kHz (optionally at 500 kHz). Aspect 89c: The composite material, method, and/or device of any preceding Aspect, wherein the composite material and/or the one or more portions thereof (having the first additive) is characterized by an ultrasound attenuation coefficient of selected from the range of 0.15 dB/mm (optionally 0.5 dB/mm) to 10 dB/mm (optionally 5 dB/mm), wherein any value and range therebetween inclusively is explicitly contemplated. Aspect 89d: The composite material, method, and/or device of any preceding Aspect, wherein the composite material and/or the one or more portions thereof (having the first additive) is characterized by an ultrasound attenuation coefficient of selected from the range of 0.05 dB/mm (optionally 0.08 dB/mm) to 10 dB/mm, and wherein any value and range therebetween inclusively is explicitly contemplated.

Aspect 90a: The composite material, method, and/or device of any preceding Aspect, wherein T_{cm,trans} is selected from the range of 25° C. to 100° C. Aspect 90a: The composite material, method, and/or device of any preceding Aspect, wherein T_{cm,trans} is selected from the range of 25° C. (optionally 26° C., optionally 27° C., optionally 28° C., optionally 29° C., optionally 30° C., optionally 31° C., optionally 32° C., optionally 33° C., optionally 34° C., optionally 35° C., optionally 36° C., optionally 37° C., optionally 38° C., optionally 39° C., optionally 40° C.) to 150° C. (optionally 40° C., optionally 45° C., optionally 50° C., optionally 55° C., optionally 60° C., optionally 65° C., optionally 70° C., optionally 75° C., optionally 80° C., optionally 85° C., optionally 90° C., optionally 95° C., optionally 100° C., optionally 110° C., optionally 120° C., optionally 130° C., optionally 140° C.), wherein any value and range therebetween inclusively is explicitly contemplated.

Aspect 92: The composite material, method, and/or device of any preceding Aspect, wherein T_{cm,trans} is the melt transition temperature (Tₘ) of the composite material.

Aspect 93: The composite material, method, and/or device of any preceding Aspect, wherein shape change is an expansion, a contraction, a twisting, an unraveling, a curling, an unfurling, an opening, a closing, a bending, an unbending, a folding, an unfolding, a straightening, a lengthening, a shortening, a redistribution or change in distribution of stress in the material, a redistribution or change in distribution of strain in the material, or any combination of these.

Aspect 94: The composite material, method, and/or device of any preceding Aspect, wherein the shape change occurs as a result of exposure of the composite material or the one or more portions thereof to ultrasound characterized by frequencies selected from the range of approximately 300 kHz (optionally within 20% thereof) to approximately 3 MHz (optionally within 20% thereof) and an energy intensity selected from the range of approximately 1 W/cm² (optionally within 20% thereof) to approximately 3 W/cm² (optionally within 20% thereof).

Aspect 95a: The composite material, method, and/or device of any preceding Aspect, wherein the shape change occurs and/or is complete within 300 seconds (optionally 260 seconds, optionally 240 seconds, optionally 200 seconds, optionally 180 seconds, optionally 150 seconds, optionally 120 seconds, optionally 100 seconds, optionally 60 seconds, optionally 45 seconds, optionally 30 seconds, optionally 15 seconds) of exposure of the composite material or the one or more portions thereof to ultrasound characterized by frequencies selected from the range of approximately 300 kHz to approximately 3 MHz and an energy intensity selected from the range of approximately 1 W/cm² to approximately 3 W/cm². Aspect 96b: The composite material, method, and/or device of any preceding Aspect, wherein the shape change occurs and/or is complete within 300 seconds (optionally 260 seconds, optionally 240 seconds, optionally 200 seconds, optionally 180 seconds, optionally 150 seconds, optionally 120 seconds, optionally 100 seconds, optionally 60 seconds, optionally 45 seconds, optionally 30 seconds, optionally 15 seconds) of exposure of the composite material or the one or more portions thereof to ultrasound.

Aspect 97a: The composite material, method, and/or device of any preceding Aspect, wherein the one or more portions having the first additive exhibit heating at a rate selected from the range of 0.1° C./s to 5° C./s, wherein any value and range therebetween inclusively is explicitly contemplated, with exposure to ultrasound characterized by frequencies selected from the range of approximately 300 kHz to approximately 3 MHz and an energy intensity selected from the range of approximately 1 W/cm² to approximately 3 W/cm². Aspect 97b: The composite material, method, and/or device of any preceding Aspect, wherein the one or more portions having the first additive exhibit an average heating rate selected from the range of 0.1° C./s to 5° C./s (where average heating rate corresponds to ΔT/timeₑₓₚₒₛᵤᵣₑ, where ΔT is the change in temperature from beginning of test to maximum temperature reached and timeₑₓₚₒₛᵤᵣₑ is the corresponding time of ultrasound exposure) with exposure to ultrasound characterized by frequencies selected from the range of approximately 300 kHz to approximately 3 MHz and an energy intensity selected from the range of approximately 1 W/cm² to approximately 3 W/cm². Aspect 97c: The composite material, method, and/or device of any preceding Aspect, wherein the one or more portions having the first additive exhibit an average heating rate selected from the range of 0.1° C./s (optionally 0.15° C./s, optionally 0.2 C/s, optionally 0.25 C/s, optionally 0.3 C/s,) to 5° C./s (optionally 0.35 C/s, optionally 0.36 C/s, optionally 0.37 C/s, optionally 0.39 C/s, optionally 0.40 C/s, optionally 0.42 C/s, optionally 0.45 C/s, optionally 0.47 C/s, optionally 0.49° C./s) (where average heating rate corresponds to ΔT/timeₑₓₚₒₛᵤᵣₑ, where ΔT is the change in temperature from beginning of test to maximum temperature reached and timeₑₓₚₒₛᵤᵣₑ is the corresponding time of ultrasound exposure) with exposure to ultrasound characterized by frequencies selected from the range of approximately 300 kHz to approximately 3 MHz and an energy intensity selected from the range of approximately 1 W/cm² to approximately 3 W/cm². Aspect 97d: The composite material, method, and/or device of any preceding Aspect, wherein the one or more portions having the first additive exhibit an average heating rate selected from the range of 0.1° C./s (optionally 0.15° C./s, optionally 0.2 C/s, optionally 0.25 C/s, optionally 0.3 C/s,) to 5° C./s (optionally 0.35 C/s, optionally 0.36 C/s, optionally 0.37 C/s, optionally 0.39 C/s, optionally 0.40 C/s, optionally 0.42 C/s, optionally 0.45 C/s, optionally 0.47 C/s, optionally 0.49° C./s) (where average heating rate corresponds to ΔT/timeₑₓₚₒₛᵤᵣₑ, where ΔT is the change in temperature from beginning of test to maximum temperature reached and timeₑₓₚₒₛᵤᵣₑ is the corresponding time of ultrasound exposure) with exposure to ultrasound.

Aspect 97a: The composite material, method, and/or device of any preceding Aspect, wherein the composite material is characterized by a density selected from the range of 0.01 to 22.5 g/cm³, and wherein any value and range therebetween inclusively is explicitly contemplated. Aspect 97a: The composite material, method, and/or device of any preceding Aspect, wherein the composite material is characterized by a Young's modulus selected from the range of 1.0 MPa to 1000 MPa at NTP, and wherein any value and range therebetween inclusively is explicitly contemplated, such as optionally 10 MPa to 100 MPa at NTP.

Aspect 98a: The composite material, method, and/or device of any preceding Aspect, wherein the first additive comprises a plurality of organic particles. Aspect 98b: The composite material, method, and/or device of any preceding Aspect, wherein the first additive comprises a plurality of inorganic particles. Aspect 98c: The composite material, method, and/or device of any preceding Aspect, wherein the first additive comprises a plurality of organic particles and a plurality of inorganic particles.

Aspect 99a: The composite material, method, and/or device of any preceding Aspect, wherein the first additive in the composite material is characterized by an ultrasound attenuation coefficient selected from the range of 0.05 dB/mm to 10 dB/mm. Aspect 99c: The composite material, method, and/or device of any preceding Aspect, wherein the first additive is an ultrasound-absorbing material characterized by an ultrasound attenuation coefficient of at least 0.16 dB/mm (optionally at least 0.17 dB/mm, optionally at least 0.18 dB/mm, optionally at least 0.19 dB/mm, optionally at least 0.20 dB/mm, optionally at least 0.21 dB/mm, optionally at least 0.22 dB/mm, optionally at least 0.23 dB/mm, optionally at least 0.24 dB/mm, optionally at least 0.25 dB/mm, optionally at least 0.26 dB/mm, optionally at least 0.27 dB/mm, optionally at least 0.28 dB/mm, optionally at least 0.29 dB/mm, optionally at least 0.30 dB/mm, optionally at least 0.31 dB/mm, optionally at least 0.32 dB/mm, optionally at least 0.33 dB/mm, optionally at least 0.34 dB/mm, optionally at least 0.35 dB/mm, optionally at least 0.37 dB/mm, optionally at least 0.40 dB/mm, optionally at least 0.42 dB/mm, optionally at least 0.45 dB/mm, optionally at least 0.47 dB/mm, optionally at least 0.50 dB/mm, optionally at least 0.51 dB/mm, optionally at least 0.52 dB/mm, optionally at least 0.53 dB/mm, optionally at least 0.54 dB/mm, optionally at least 0.55 dB/mm, optionally at least 0.56 dB/mm, optionally at least 0.57 dB/mm, optionally at least 0.58 dB/mm, optionally at least 0.59 dB/mm, optionally at least 0.60 dB/mm, optionally at least 0.65 dB/mm, optionally at least 0.70 dB/mm, optionally at least 0.75 dB/mm, optionally at least 0.80 dB/mm, optionally at least 0.85 dB/mm, optionally at least 0.9 dB/mm, optionally at least 1.0 dB/mm, optionally at least 1.2 dB/mm, optionally at least 1.5 dB/mm, optionally at least 1.75 dB/mm, optionally at least 2.0 dB/mm, optionally at least 2.25 dB/mm, optionally at least 2.50 dB/mm, optionally at least 2.75 dB/mm, optionally at least 3.0 dB/mm, optionally at least 3.25 dB/mm, optionally at least 3.5 dB/mm, optionally at least 3.75 dB/mm) at ultrasound frequencies selected from the range of 400 kHz to 600 kHz (optionally at 500 kHz). Aspect 99d: The composite material, method, and/or device of any preceding Aspect, wherein the first additive is an ultrasound-absorbing material characterized by an ultrasound attenuation coefficient of selected from the range of 0.15 dB/mm (optionally 0.5 dB/mm) to 10 dB/mm (optionally 5 dB/mm), wherein any value or range therebetween inclusively is explicitly contemplated.

Aspect 100: The composite material, method, and/or device of any preceding Aspect, wherein the first additive comprises a plurality of hollow particles (e.g., microspheres).

Aspect 101: The composite material, method, and/or device of any preceding Aspect, wherein the first additive comprises a plurality of non-hollow particles (e.g., microspheres).

Aspect 102: The composite material, method, and/or device of any preceding Aspect, wherein the first additive comprises a plurality of hollow glass beads (e.g., microspheres; e.g., glass bubbles), non-hollow glass beads (e.g., microspheres), or any combination thereof.

Aspect 103: The composite material, method, and/or device of any preceding Aspect, wherein the first additive comprises a plurality of salt particles, a plurality of metal oxide particles, a plurality of metal particles, a plurality of organic particles, or any combination thereof.

Aspect 104a: The composite material, method, and/or device of any preceding Aspect, wherein the first additive comprises a plurality of metal chloride particles, iron oxide particles, silica particles, silica gel particles, glass beads (e.g., glass particles, glass bubbles, or glass microspheres), metal particles (e.g., gold, platinum, iridium, silver, tungsten, or another metal having similar high density), or any combination thereof. Aspect 105b: The composite material, method, and/or device of any preceding Aspect, wherein the first additive comprises a plurality of metal chloride particles, iron oxide particles, silica particles, silica gel particles, glass beads (e.g., glass particles, glass bubbles, or glass microspheres), metal particles (e.g., gold, platinum, iridium, silver, tungsten, or another metal having similar high density, or alloys thereof, or alloys therewith; e.g., high density metal alloys), poly(tetrafluoroethylene)(PTFE) particles, or any combination thereof.

Aspect 1061a: The composite material, method, and/or device of any preceding Aspect, wherein the first additive comprises a plurality of hollow glass microspheres or particles and/or non-hollow glass microspheres or particles characterized by a median diameter selected from the range of approximately 15 μm (optionally 16 μm, optionally 17 μm, optionally 18 μm, optionally 19 μm, optionally 20 μm, optionally 21 μm, optionally 22 μm, optionally 23 μm, optionally 24 μm, optionally 25 μm, optionally 30 μm, optionally 35 μm) to approximately 1000 μm (optionally 36 μm, optionally 40 μm, optionally 45 μm, optionally 50 μm, optionally 55 μm, optionally 60 μm, optionally 65 μm, optionally 70 μm, optionally 75 μm, optionally 80 μm, optionally 85 μm, optionally 90 μm, optionally 95 μm, optionally 100 μm, optionally 200 μm, optionally 300 μm, optionally 400 μm, optionally 500 μm, optionally 600 μm, optionally 700 μm, optionally 800 μm, optionally 900 μm). Aspect 106b: The composite material, method, and/or device of any preceding Aspect, wherein the first additive comprises a plurality of hollow glass microspheres or particles and/or non-hollow glass microspheres or particles characterized by an average diameter selected from the range of approximately 15 μm (optionally 16 μm, optionally 17 μm, optionally 18 μm, optionally 19 μm, optionally 20 μm, optionally 21 μm, optionally 22 μm, optionally 23 μm, optionally 24 μm, optionally 25 μm, optionally 30 μm, optionally 35 μm) to approximately 1000 μm (optionally 36 μm, optionally 40 μm, optionally 45 μm, optionally 50 μm, optionally 55 μm, optionally 60 μm, optionally 65 μm, optionally 70 μm, optionally 75 μm, optionally 80 μm, optionally 85 μm, optionally 90 μm, optionally 95 μm, optionally 100 μm, optionally 200 μm, optionally 300 μm, optionally 400 μm, optionally 500 μm, optionally 600 μm, optionally 700 μm, optionally 800 μm, optionally 900 μm), wherein any value or range therebetween inclusively is explicitly contemplated. Aspect 106c: The composite material, method, and/or device of any preceding Aspect, wherein the first additive comprises a plurality of hollow particles and/or non-hollow particles characterized by a median diameter selected from the range of approximately 15 μm (optionally 16 μm, optionally 17 μm, optionally 18 μm, optionally 19 μm, optionally 20 μm, optionally 21 μm, optionally 22 μm, optionally 23 μm, optionally 24 μm, optionally 25 μm, optionally 30 μm, optionally 35 μm) to approximately 1000 μm (optionally 36 μm, optionally 40 μm, optionally 45 μm, optionally 50 μm, optionally 55 μm, optionally 60 μm, optionally 65 μm, optionally 70 μm, optionally 75 μm, optionally 80 μm, optionally 85 μm, optionally 90 μm, optionally 95 μm, optionally 100 μm, optionally 200 μm, optionally 300 μm, optionally 400 μm, optionally 500 μm, optionally 600 μm, optionally 700 μm, optionally 800 μm, optionally 900 μm), wherein any value or range therebetween inclusively is explicitly contemplated.

Aspect 107: The composite material, method, and/or device of any preceding Aspect, wherein the first additive comprises a plurality of hollow microspheres characterized by a median internal cavity diameter selected from the range of 1 μm (optionally 2 μm, optionally 3 μm, optionally 4 μm, optionally 5 μm, optionally 6 μm, optionally 7 μm, optionally 8 μm, optionally 9 μm, optionally 10 μm, optionally 11 μm, optionally 12 μm, optionally 13 μm, optionally 14 μm, optionally 15 μm, optionally 20 μm) to 990 μm (optionally 6 μm, optionally 7 μm, optionally 8 μm, optionally 9 μm, optionally 10 μm, optionally 11 μm, optionally 12 μm, optionally 13 μm, optionally 14 μm, optionally 15 μm, optionally 20 μm, optionally 25 μm, optionally 30 μm, optionally 50 μm, optionally 100 μm, optionally 150 μm, optionally 200 μm) and/or an average wall thickness selected from the range of 0.2 μm (optionally 0.3 μm, optionally 0.4 μm, optionally 0.5 μm, optionally 0.6 μm, optionally 0.7 μm, optionally 0.8 μm, optionally 0.9 μm, optionally 1 μm) to 5 μm (optionally 1.2 μm, optionally 1.3 μm, optionally 1.4 μm, optionally 1.5 μm, optionally 2 μm, optionally 3 μm, optionally 4 μm, optionally 4.5 μm), wherein any value or range therebetween inclusively is explicitly contemplated.

Aspect 108a: The composite material, method, and/or device of any preceding Aspect, wherein a concentration of the first additive in the one or more shape memory polymers is selected from the range of 0.4 wt. % (optionally 0.5 wt. %, optionally 0.6 wt. %, optionally 0.7 wt. %, optionally 0.8 wt. %, optionally 0.9 wt. %, optionally 1.0 wt. %, optionally 1.1 wt. %, optionally 1.2 wt. %, optionally 1.5 wt. %, optionally 1.7 wt. %, optionally 2.0 wt. %, optionally 2.2 wt. %, optionally 2.5 wt. %, optionally 2.7 wt. %, optionally 3.0 wt. %, optionally 3.2 wt. %, optionally 3.5 wt. %, optionally 3.7 wt. %, optionally 4.0 wt. %, optionally 4.2 wt. %, optionally 4.5 wt. %, optionally 4.7 wt. %, optionally 5.0 wt. %) to 50 wt. % (optionally 5 wt. %, optionally 6 wt. %, optionally 7 wt. %, optionally 8 wt. %, optionally 9 wt. %, optionally 10 wt. %, optionally 11 wt. %, optionally 12 wt. %, optionally 13 wt. %, optionally 14 wt. %, optionally 15 wt. %, optionally 20 wt. %, optionally 25 wt. %, optionally 30 wt. %, optionally 35 wt. %, optionally 40 wt. %, optionally 45 wt. %), wherein any value or range therebetween inclusively is explicitly contemplated, with respect to weight of the one or more polymers in the composite material. Aspect 108b: The composite material, method, and/or device of any preceding Aspect, wherein the first additive comprises a plurality of metal oxide particles (e.g., iron oxide particles); and wherein a concentration of the first additive in the one or more shape memory polymers is selected from the range of approximately 1 wt. % (optionally 1.0 wt. %, optionally 1.1 wt. %, optionally 1.2 wt. %, optionally 1.5 wt. %, optionally 1.7 wt. %, optionally 2.0 wt. %, optionally 2.2 wt. %, optionally 2.5 wt. %, optionally 2.7 wt. %, optionally 3.0 wt. %, optionally 3.2 wt. %, optionally 3.5 wt. %, optionally 3.7 wt. %, optionally 4.0 wt. %, optionally 4.2 wt. %, optionally 4.5 wt. %, optionally 4.7 wt. %, optionally 5.0 wt. %) to 30 wt. % (optionally 5 wt. %, optionally 6 wt. %, optionally 7 wt. %, optionally 8 wt. %, optionally 9 wt. %, optionally 10 wt. %, optionally 11 wt. %, optionally 12 wt. %, optionally 13 wt. %, optionally 14 wt. %, optionally 15 wt. %), wherein any value or range therebetween inclusively is explicitly contemplated, with respect to weight of the one or more polymers in the composite material. Aspect 108c: The composite material, method, and/or device of any preceding Aspect, wherein the first additive comprises a plurality of hollow and/or non-hollow glass microspheres (e.g., K25 hollow glass microspheres; e.g., iM30K hollow glass microspheres); and wherein a concentration of the first additive in the one or more shape memory polymers is selected from the range of approximately 0.4 wt. % (optionally 0.5 wt. %, optionally 0.6 wt. %, optionally 0.7 wt. %, optionally 0.8 wt. %, optionally 0.9 wt. %, optionally 1.0 wt. %, optionally 1.1 wt. %, optionally 1.2 wt. %, optionally 1.5 wt. %) to 50 wt. % (optionally 5 wt. %, optionally 6 wt. %, optionally 7 wt. %, optionally 8 wt. %, optionally 9 wt. %, optionally 10 wt. %), wherein any value or range therebetween inclusively is explicitly contemplated, with respect to weight of the one or more polymers in the composite material.

Aspect 109a: The composite material, method, and/or device of any preceding Aspect, wherein the first additive is biologically inert and/or is substantially insoluble in a biological fluid under physiological or in-vivo conditions. Aspect 109b: The composite material, method, and/or device of any preceding Aspect, wherein the first additive is substantially insoluble in saline fluid over a time period selected from the range of 1 day to at least 6 months.

Aspect 110: The composite material, method, and/or device of any preceding Aspect, wherein the first additive or the plurality of particles thereof is characterized by a density selected from the range of 0.01 g/cm³ (optionally 0.02 g/cm³, optionally 0.03 g/cm³, optionally 0.05 g/cm³, optionally 0.07 g/cm³, optionally 0.09 g/cm³, optionally 0.1 g/cm³, optionally 1.2 g/cm³, optionally 1.5 g/cm³, optionally 1.7 g/cm³, optionally 2.0 g/cm³, optionally 2.5 g/cm³) to 22.5 g/cm³ (optionally 0.8 g/cm³, optionally 1.0 g/cm³, optionally 1.5 g/cm³, optionally 2.0 g/cm³, optionally 2.5 g/cm³, optionally 3.0 g/cm³, optionally 3.5 g/cm³, optionally 4.0 g/cm³, optionally 4.5 g/cm³, optionally 5.0 g/cm³, optionally 5.2 g/cm³, optionally 5.5 g/cm³, optionally 6.0 g/cm³, optionally 6.5 g/cm³), optionally 0.1 g/cm³ to 5.2 g/cm³, wherein any value or range therebetween inclusively is explicitly contemplated.

Aspect 111: The composite material, method, and/or device of any preceding Aspect, wherein the first additive or the plurality of particles thereof is characterized by a heat capacity selected from the range of 0.10 JC⁻¹g⁻¹ (optionally 0.11 JC⁻¹g⁻¹, optionally 0.15 JC⁻¹g⁻¹, optionally 0.20 JC⁻¹g⁻¹, optionally 0.25 JC⁻¹g⁻¹, optionally 0.30 JC⁻¹g⁻¹, optionally 0.5 JC⁻¹g⁻¹, optionally 0.65 JC⁻¹g⁻¹, optionally 0.7 JC⁻¹g⁻¹, optionally 0.75 JC⁻¹g⁻¹, optionally 0.8 JC⁻¹g⁻¹, optionally 0.9 JC⁻¹g⁻¹, optionally 1.0 JC⁻¹g⁻¹, optionally 1.5 JC⁻¹g⁻¹) to 4.5 JC⁻¹g⁻¹ (optionally 1.0 JC⁻¹g⁻¹, optionally 1.5 JC⁻¹g⁻¹, optionally 2.0 JC⁻¹g⁻¹, optionally 2.5 JC⁻¹g⁻¹, optionally 3.0 JC⁻¹g⁻¹, optionally 3.5 JC⁻¹g⁻¹, optionally 4.0 JC⁻¹g⁻¹, optionally 4.2 JC⁻¹g⁻¹, optionally 4.5 JC⁻¹g⁻¹, optionally 5.0 JC⁻¹g⁻¹), optionally 0.7 JC⁻¹g⁻¹ to optionally 1.0 JC⁻¹g⁻¹, wherein any value or range therebetween inclusively is explicitly contemplated.

Aspect 112: The composite material, method, and/or device of any preceding Aspect, wherein the first additive or the plurality of particles thereof is characterized by a thermal conductivity selected from the range of 0.02 Wm⁻¹K⁻¹ (optionally 0.03 Wm⁻¹K⁻¹, optionally 0.04 Wm⁻¹K⁻¹, optionally 0.05 Wm⁻¹K⁻¹, optionally 0.08 Wm⁻¹K⁻¹, optionally 0.10 Wm⁻¹K⁻¹, optionally 0.15 Wm⁻¹K⁻¹, optionally 0.20 Wm⁻¹K⁻¹, optionally 0.25 Wm⁻¹K⁻¹, optionally 0.5 Wm⁻¹K⁻¹, optionally 0.75 Wm⁻¹K⁻¹, optionally 1.0 Wm⁻¹K⁻¹, optionally 1.25 Wm⁻¹K⁻¹, optionally 1.5 Wm⁻¹K⁻¹) to 500.00 Wm⁻¹K⁻¹ (optionally 2 Wm⁻¹K⁻¹, optionally 3 Wm⁻¹K⁻¹, optionally 5 Wm⁻¹K⁻¹, optionally 6 Wm⁻¹K⁻¹, optionally 9 Wm⁻¹K⁻¹, optionally 10 Wm⁻¹K⁻¹, optionally 15 Wm⁻¹K⁻¹, optionally 50 Wm⁻¹K⁻¹, optionally 100 Wm⁻¹K⁻¹, optionally 200 Wm⁻¹K⁻¹, optionally 300 Wm⁻¹K⁻¹, optionally 400 Wm⁻¹K⁻¹, optionally 428 Wm⁻¹K⁻¹, optionally 500 Wm⁻¹K⁻¹), optionally 0.04 Wm⁻¹K⁻¹ to 6.0 Wm⁻¹K⁻¹, wherein any value or range therebetween inclusively is explicitly contemplated.

Aspect 113: The composite material, method, and/or device of any preceding Aspect, wherein the first additive or the plurality of particles thereof is characterized by a density selected from the range of 0.1 to 0.6 g/cm³, wherein any value or range therebetween inclusively is explicitly contemplated, such as optionally 0.25 to 0.6 g/cm³.

Aspect 114: The composite material, method, and/or device of any preceding Aspect, wherein the first additive or the plurality of particles thereof is characterized by a thermal conductivity selected from the range of 0.04 to 0.20 Wm⁻¹K⁻¹, wherein any value or range therebetween inclusively is explicitly contemplated.

Aspect 115a: The composite material, method, and/or device of any preceding Aspect, wherein the first additive is a plurality of particles having characteristic sizes selected from the range of 0.030 to 1000 μm, wherein any value or range therebetween inclusively is explicitly contemplated, such as optionally 15 μm to 55 μm. Aspect 115b: The composite material, method, and/or device of any preceding Aspect, wherein the first additive is a plurality of particles characterized by a median size selected from the range of 0.030 to 1000 μm, wherein any value or range therebetween inclusively is explicitly contemplated, such as optionally 15 μm to 55 μm. Aspect 115c: The composite material, method, and/or device of any preceding Aspect, wherein the first additive is a plurality of particles characterized by an average size selected from the range of 0.030 μm to 1000 μm, wherein any value or range therebetween inclusively is explicitly contemplated, such as optionally 15 μm to 55 μm.

Aspect 115d: The composite material, method, and/or device of any preceding Aspect, wherein the first additive comprises a plurality of particles having a median characteristic size selected from the range of 0.030 to 1000 μm, wherein any value or range therebetween inclusively is explicitly contemplated, such as optionally 15 μm to 55 μm.

Aspect 116: The composite material, method, and/or device of any preceding Aspect, wherein one or more additive-free crosslinked shape memory polymers equivalent to the one or more shape memory polymers of the composite material are characterized by a polymer-only transition temperature (T_{pol,trans}), and wherein T_{cm,trans} deviates from T_{pol,trans} by no more than 5° C. (optionally no more than 4° C., optionally no more than 3° C., optionally no more than 2° C., optionally no more than 1.5° C., optionally no more than 1° C.) and/or no more than 10% (optionally no more than 8%, optionally no more than 5%). For example, Tₘ varies by no more than 1.5° C. with vs. without additives where the additives are 5 wt. % HGMs or SGMs (MK⁻¹-39, 8 examples, DBzP as crosslinker). For example, Tₘ varies by no more than 2.0° C. with vs. without additives, where additives are HGMs or SGMs (MK⁻¹-21, 12 examples, DCP as crosslinker, varying wt. % 0.5-5.0). With Fe₃O₄, large variations in Tₘ were seen at 5.0 and 15.0 wt. %. For example, Tₘ varies by no more than 1.9° C. with vs. without additives, where additives are NaCl, silica gel, K25 HGMs, Fe₃O₄ NPs, are used at 1.0 wt. % (MK1-7, 8 examples, di-COE as crosslinker).

Aspect 117: The composite material, method, and/or device of any preceding Aspect, wherein the one or more shape memory polymers is characterized by an upper limit of crosslinking temperature of at least 65° C. (optionally at least 70° C., optionally at least 75° C., optionally at least 80° C., optionally at least 85° C., optionally at least 90° C., optionally at least 95° C., optionally at least 100° C., optionally at least 105° C., optionally at least 110° C., optionally at least 115° C., optionally at least 120° C., optionally at least 125° C., optionally at least 130° C.).

Aspect 118: The composite material, method, and/or device of any preceding Aspect, wherein the one or more shape memory polymers comprise crosslinking moieties derived from a crosslinking precursor is one or more organic peroxide compounds having a 10-hour half-life temperature (HLT) at least 10° C. greater (optionally at least 5° C. greater, optionally at least 12° C. greater, optionally at least 15° C. greater) than a melt temperature (Tₘ) and/or the T_{pol,trans} of the one or more shape memory polymers.

Aspect 119: The composite material, method, and/or device of any preceding Aspect, wherein the one or more shape memory polymers comprise a crosslinking moieties derived from a crosslinking precursor selected from the group consisting of: a di(4-cyclooctenol) succinate, dicumyl peroxide (DCP), dibenzoyl peroxide (DBzP), di(tert-butyl) peroxide, and any combination thereof.

Aspect 120a: The composite material, method, and/or device of any preceding Aspect, wherein one or more shape memory polymers comprise a poly(cyclooctene), a polycaprolactone, a poly(lactic acid), a poly(lactic-co-glycolic acid), a polyethylene, a polypropylene, a thermoplastic polyurethane (TPU), or any combination thereof. Aspect 38b: The composite material, method, and/or device of any preceding Aspect, wherein the one or more shape memory polymers comprise a poly(cyclooctene), a polycaprolactone, or any combination thereof.

Aspect 121: The composite material, method, and/or device of any preceding Aspect, comprising a second additive being different from the first additive.

Aspect 122a: The composite material, method, and/or device of Aspect 121, wherein the second additive comprises a plurality of inorganic particles, a plurality of hollow particles, a plurality of particles characterized by an ultrasound attenuation coefficient selected from the range of 0.05 dB/mm to 10 dB/mm, or any combination thereof. Aspect 122b: The composite material, method, and/or device of Aspect 121, wherein the second additive is according to any of the preceding Aspects but is different from the first additive.

Aspect 123: The composite material, method, and/or device of any preceding Aspect, wherein the temporary shape is compressed with respect to the permanent shape and wherein the shape change comprises expansion.

Aspect 124: A device comprising the composite material of any preceding Aspect.

Aspect 113: A method of using the composite material of any preceding Aspect, the method comprising:
directing one or more focused ultrasound beams at the one or more portions of the composite material;
thereby, heating the one or more portions to a temperature approximately equal to or greater than the T_{cm,trans}; and
thereby, causing the composite material to undergo the shape change at the one or more portions thereof.

Aspect 114: The method of Aspect 113, wherein the step of directing comprises exposing each of the one or more portions to the one or more focused ultrasound beams for a consecutive/uninterrupted time being less than 5 minutes.

Aspect 115: The method of Aspect 113 or 114, wherein the step of directing comprises controlling and varying an exposure time, power, and/or exposure area of the one or more focused ultrasound beams.

Aspect 116: The method of any of Aspects 113-115, wherein the step of directing comprises exposing the one or more portions to different focused ultrasound beams characterized by different exposure time, power, and/or exposure area.

Aspect 117: The method of any of Aspects 113-116 comprising actuating an actuator or switch of a device; wherein the actuator or switch comprises the composite material; and wherein the shape change causes the actuating.

Aspect 118a: The method of any of Aspects 113-117 comprising setting the temporary shape of the composite material at a temperature equal to or greater than T_{cm,trans} and less than a crosslinking temperature (T_{cm,crosslink}) of the composite material. Aspect 118b: The method of any of Aspects 113-117 comprising setting the temporary shape of the composite material at a temperature equal to or greater than T_{cm,trans} and maintaining said temperature as the composite material is cooled to below T_{cm,trans}. Aspect 118c: The method of any of Aspects 113-117 comprising setting the temporary shape of the composite material at a temperature equal to or greater than T_{cm,trans}.

Aspect 119: A method of making the composite material of any of the preceding Aspects, the method comprising:
polymerizing a monomer to form a first polymer;
crosslinking the first polymer in the presence of a crosslinking precursor and the first additive at a temperature approximately (e.g., within 20%) equal to or greater than a crosslinking temperature (T_{cm,crosslink}) (optionally at a temperature greater than or equal to 65° C., optionally a temperature greater than or equal to 75° C., a temperature greater than or equal to 85° C., a temperature greater than or equal to 90° C., a temperature greater than or equal to 95° C., a temperature greater than or equal to 100° C., a temperature greater than or equal to 105° C., a temperature greater than or equal to 110° C., a temperature greater than or equal to 115° C.) to form the composite material having the crosslinked shape memory polymer and the first additive.

Aspect 120: The method of Aspect 119, wherein the first additive is provided with the monomer and the step of polymerizing is performed in the presence of the first additive such that the first polymer comprises the first additive.

Aspect 121: The method of Aspect 119, wherein the step of polymerization is performed in absence of the first additive and the first additive is provided to the step of crosslinking.

Aspect 122: The method of any of Aspects 119-121, wherein the step of crosslinking is performed separately after the step of polymerizing.

Aspect 123: The method of any of Aspects 119-122, wherein the steps of polymerizing and crosslinking are performed substantially concurrently as one step.

Aspect 124: The method of any of Aspects 119-123, wherein the crosslinking precursor is selected from the group consisting of: a di(4-cyclooctenol) succinate, dicumyl peroxide (DCP), dibenzoyl peroxide (DBzP), di(tert-butyl) peroxide, and any combination thereof.

Aspect 125: The method of any of Aspects 119-124, wherein the monomer is selected from the group consisting of: cyclooctene, cyclopentene, cycloheptene, butadiene, and any combination thereof.

Aspect 126: The method of any of Aspects 119-125, comprising selecting the first additive and a concentration thereof to tune the T_{cm,trans} and one or more ultrasound-absorption characteristics of the resulting composite material.

Aspect 127: The method of any of Aspects 119-126, wherein the step of crosslinking comprises setting the permanent shape of the composite material at a temperature equal to or greater than the crosslinking temperature (T_{cm,crosslink}) for a crosslinking time period.

Aspect 128: The method of Aspect 127, wherein the step of setting the permanent shape comprises confining the composite material, molding the composite material, or otherwise applying a mechanical force to the composite material while the composite material or at least a portion thereof is at a temperature equal to or greater than the crosslinking temperature (T_{cm,crosslink}).

Aspect 129: The method of any of Aspects 127-128, wherein an upper limit of crosslinking temperature of the composite material is at least 65° C.

Aspect 130a: The method of any of Aspects 119-129 comprising setting the temporary shape of the composite material at a temperature within 35° C. of T_{cm,trans} or a temperature approximately equal to or greater than T_{cm,trans} and less than a crosslinking temperature (T_{cm,crosslink}) or an upper limit of crosslinking temperature of the composite material. Aspect 130b: The method of any of Aspects 119-129 comprising setting the temporary shape of the composite material at a temperature equal to or greater than T_{cm,trans} and maintaining said temperature as the composite material is cooled to below T_{cm,trans}. Aspect 130c: The method of any of Aspects 119-129 comprising setting the temporary shape of the composite material at a temperature equal to or greater than T_{cm,trans}.

Aspect 131: The method of Aspect 130, wherein the step of setting the temporary shape comprises confining the composite material, molding the composite material, or otherwise applying a mechanical force to the composite material while is composite material or at least a portion thereof is at a temperature equal to or greater than T_{cm,trans}.

Aspect 132: A method of making the device of any of the preceding claims, the method comprising:
attaching, providing, or inserting the composite material of any of the preceding Aspects to or into the device.

Aspect 133: The method of Aspect 132, comprising setting the temporary shape of the composite material at a temperature within 35° C. of T_{cm,trans} or a temperature approximately equal to or greater than T_{cm,trans} and less than a crosslinking temperature (T_{cm,crosslink}) or an upper limit of crosslinking temperature of the composite material. Aspect 133b: The method of any of Aspect 132 comprising setting the temporary shape of the composite material at a temperature equal to or greater than T_{cm,trans} and maintaining said temperature as the composite material is cooled to below T_{cm,trans}. Aspect 132c: The method of any of Aspect 132 comprising setting the temporary shape of the composite material at a temperature equal to or greater than T_{cm,trans}.

Aspect 134: The method of Aspect 133, wherein the step of setting the temporary shape comprises confining the composite material, molding the composite material, or otherwise applying a mechanical force to the composite material while is composite material or at least a portion thereof is at a temperature equal to or greater than T_{cm,trans}.

Aspect 135: The method of any of Aspects 132-134, wherein the step of setting the temporary shape is performed prior to attaching, providing, or inserting the composite material to or into the device.

Aspect 136: A method of making the device of any of the preceding claims, the method comprising: shaping or forming the composite material of any of the preceding Aspects thereby forming the device, the device being substantially formed of the composite material.

Aspect 137a: The composite material, method, and/or device of any preceding Aspect having one shape memory polymer. Aspect 137b: The composite material, method, and/or device of any preceding Aspect having two or more shape memory polymers.

Aspect 138: The composite material, method, and/or device of any preceding Aspect, wherein the shape change is from the temporary shape of the composite material an intermediate shape, the intermediate shape being a shape at some point between the temporary shape and the original permanent shape but before or without obtaining the original permanent shape.

Aspect 139: The method of any preceding Aspect comprising providing the first additive at only one or more portions of the shape memory polymer(s) such that the first additive is non-uniformly or non-homogeneously provided in the shape memory polymer(s).

Aspect 140a: The composite material, method, and/or device of any preceding Aspect, wherein the first additive is non-uniformly or non-homogeneously present in the shape memory polymer(s). Aspect 140b: The composite material, method, and/or device of any preceding Aspect, wherein the first additive is uniformly or homogeneously present in the shape memory polymer(s).

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Certain molecules disclosed herein may contain one or more ionizable groups [groups from which a proton can be removed (e.g., —COON) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every device, component, feature, system, formulation, combination of components, or method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. An implantable, detachable, and removable medical device comprising:
a therapeutic balloon configured to perform a therapeutic activity inside a living subject; and
a focused-ultrasound activatable actuator operably connected to the therapeutic balloon;
wherein the actuator is capable of being activated between a closed state and an open state remotely via a focused ultrasound beam;
wherein the device is configured to be in an implantation state to facilitate implantation of the device in the living subject, in a therapeutic state to facilitate performance of the therapeutic activity, and subsequently in an expulsion state to facilitate expulsion of the medical device from the living subject; and
wherein the implantation state, therapeutic state, and expulsion state are different from each other.

2. The medical device of claim 1, wherein the actuator is capable of being activated remotely via a focused ultrasound beam.

3. (canceled)

4. The medical device of claim 1, wherein the therapeutic state comprises the actuator being in the closed state and the expulsion state comprises the actuator being in the open state.

5. The medical device of claim 1, wherein the implantation state and therapeutic state are characterized by the actuator being in its closed state; and wherein activation of the actuator from its closed state to its open state activates the expulsion state of the device.

6. The medical device of claim 1, wherein the device is in the implantation state when being implanted in the living subject, subsequently in the therapeutic state when performing the therapeutic activity in the living subject, and further subsequently in the expulsion state when being expelled from the living subject.

7. The medical device of claim 1, wherein the therapeutic state is characterized by the therapeutic balloon having a larger volume and/or cross-sectional size than the same of the expulsion state of the device.

8. The medical device of claim 7, wherein the therapeutic state is characterized by the therapeutic balloon being expanded or inflated and the expulsion state is characterized by the therapeutic balloon being contracted or deflated.

9. The medical device of claim 1, wherein the implantation state is characterized by the medical device comprising an implant-accessory that is operably and detachably connected to the therapeutic component; wherein the implant-accessory is capable of providing a fluid into a lumen of the therapeutic balloon; and wherein the implant accessory is detachable and removable without deflating the therapeutic balloon upon detachment and removal of the implant accessory.

10. The medical device of claim 9, wherein the expulsion state is characterized by the medical device being free of and not operably connected to the implant-accessory.

11. The medical device of claim 9, wherein the implant-accessory facilitates providing the medical device to a desired region of the living subject and the implant-accessory facilitates converting the device from the implantation state to the therapeutic state.

12. The medical device of claim 11 being configured to be disconnected from the implant-accessory after the therapeutic state of the device is activated.

13. The medical device of claim 8, wherein the therapeutic state comprises the therapeutic balloon being inflated with a biologically-benign fluid; and wherein the expulsion state comprises the therapeutic balloon being deflated as a result of being mostly free of the biologically-benign fluid or as a result of containing less of the biologically-benign fluid than in the therapeutic state.

14. The medical device of claim 13, wherein the therapeutic state comprises the therapeutic balloon remaining inflated substantially only by a fluid pressure of the biologically-benign fluid inside the therapeutic balloon.

15. The medical device of claim 13, wherein the therapeutic state comprises the therapeutic balloon being fluidically sealed.

16. (canceled)

17. The medical device of claim 1, wherein the actuator comprises a polymer component capable of absorbing focused ultrasound; wherein one or more characteristics of the polymer component change as a result of it absorbing the focused ultrasound; and wherein the actuator is activated as a result of the change of the one or more characteristics of the polymer component; and wherein the polymer component comprises a shape change polymer capable of absorbing focused ultrasound; wherein the shape change polymer is at least partially heated by absorbing the focused ultrasound as a result of which the polymer component undergoes an ultrasound-induced shape change.

18. (canceled)

19. The medical device of claim 17, wherein the polymer component is characterized by a component transition temperature (T_{cm,trans}); wherein the polymer component or one or more portions thereof undergo the shape change from a temporary shape to a permanent shape when the polymer component or said one or more portions thereof are heated to within 35° C. of T_{cm,trans} or a temperature approximately equal to or greater than T_{cm,trans}; wherein T_{cm,trans} is selected from the range of 40° C. to 100° C.

20. (canceled)

21. (canceled)

22. The medical device of claim 17, wherein the ultrasound-induced shape change occurs as a result of exposure of the composite material or the one or more portions thereof to ultrasound characterized by frequencies selected from the range of approximately 300 kHz to approximately 3 MHz and an energy intensity selected from the range of approximately 1 W/cm² to approximately 3 W/cm²; and wherein the polymer component is an ultrasound-absorbing material characterized by an ultrasound attenuation coefficient selected from the range of 0.05 dB/mm to 10 dB/mm.

23. (canceled)

24. The medical device of claim 17, wherein shape memory polymer comprises polycyclooctene (PCOE), polycaprolactone (PCL), poly(lactic acid)(PLA), poly(lactic-co-glycolic acid)(PLGA), polyethylene (PE), polypropylene (PP), and thermoplastic polyurethane (TPU), or any combination thereof; and wherein the shape memory polymer comprises a crosslinking moiety derived from a crosslinking precursor selected from the group consisting of: a di(4-cyclooctenol) succinate, dicumyl peroxide (DCP), dibenzoyl peroxide (DBzP), di(tert-butyl) peroxide, and any combination thereof.

25. (canceled)

26. The medical device of claim 17, wherein the actuator is a valve for retaining a biologically-benign fluid in the therapeutic balloon or for releasing a biologically-benign fluid from the therapeutic balloon.

27. The medical device of claim 26, wherein the open state of the actuator is configured to allow the biologically-benign fluid to pass therethrough and the closed state of the actuator is configured to block passage of the biologically-benign fluid therethrough.

28. The medical device of claim 26, wherein the actuator comprises a fluid-escape conduit and the polymer component is a plug; wherein the closed state of the actuator is characterized by the plug blocking fluid flow through the fluid-escape conduit, thereby retaining the biologically-benign fluid in the therapeutic balloon; and wherein the therapeutic state comprises the actuator being in the closed state; and wherein the ultrasound-induced shape change of the polymer component activates the open state of the actuator.

29. (canceled)

30. (canceled)

31. (canceled)

32. (canceled)

33. The medical device of claim 28, wherein the ultrasound-induced shape change is an expansion of the plug such that the plug is at least partially released from the fluid-escape conduit allowing the biologically-benign fluid to flow through the fluid-escape conduit.

34. (canceled)

35. The medical device of claim 28, wherein the ultrasound-induced shape change is a decrease in cross-sectional dimension of the plug from at least equal to or greater than a diameter of the fluid-escape conduit to less than the diameter of the fluid-escape conduit.

36. (canceled)

37. The medical device of claim 28, wherein the ultrasound-induced shape change comprises formation of a hole or opening in the plug through which the biologically-benign fluid may flow.

38. (canceled)

39. The medical device of claim 28, wherein the ultrasound-induced shape change comprises an unfolding which forms a fluid pathway.

40. (canceled)

41. The medical device of claim 27, wherein the actuator and the polymer component thereof are configured to remain physically connected to or a part of the medical device in the implantation state, in the therapeutic state, and in the expulsion state.

42. The medical device of claim 1, wherein the therapeutic state and expulsion state are characterized by the device being free of and physically disconnected from any component that is at least partially external to the living subject.

43. The medical device of claim 1 being configured to remain implanted in the living subject in the therapeutic state for at least 12 hours.

44. The medical device of claim 1, wherein the therapeutic state is characterized by the therapeutic balloon having an inflated diameter selected from the range of 6 mm to 12 mm; wherein the therapeutic state is characterized by the therapeutic balloon having a length selected from the range of 10 mm to 30 mm; and wherein the expulsion state is characterized by the therapeutic balloon having a deflated diameter selected from the range of 0.5 mm to 2 mm.

45. (canceled)

46. (canceled)

47. (canceled)

48. (canceled)

49. The medical device of claim 1, wherein the device is configured to be self-expelled via a natural bodily process of the living subject and does not require an invasive removal procedure.

50. The medical device of claim 1, wherein the device is an occlusion device and the therapeutic activity is an occlusion.

51. (canceled)

52. (canceled)

53. (canceled)

54. The medical device of claim 50, wherein the living subject is an infant or a fetus.

55. (canceled)

56. (canceled)

57. (canceled)

58. (canceled)

59. (canceled)

60. (canceled)

61. The medical device of claim 1 being free of electronics, a sensor, a battery, electrical wires, or any combination thereof.

62. A method for treating a condition in a living subject, the method comprising:
implanting the medical device of any one of the preceding claims with the aid of an implant-accessory;
filling the therapeutic balloon with a biologically-benign fluid via the implant-accessory; and
detaching the implant-accessory from the medical device.

63-69. (canceled)
